(19) United States
(12) Patent Application Publication
(10) Pub. No.: US 2024/0336807 A1
(43) Pub. Date: Oct. 10, 2024

(54) COATINGS, FORMULATIONS, USES AND COATING METHODS

(71) Applicant: BIOINTERACTIONS LTD, Shinfield Reading (GB)

(72) Inventors: Sajinder Kaur LUTHRA, Shinfield Reading (GB); Arjun Kamal Singh LUTHRA, Shinfield Reading (GB); Arundeep Singh LUTHRA, Shinfield Reading (GB)

(73) Assignee: BIOINTERACTIONS LTD, Shinfield Reading (GB)

(21) Appl. No.: 18/579,412

(22) PCT Filed: Jul. 18, 2022
(86) PCT No.: PCT/GB2022/051848

§ 371 Date: Jan. 15, 2024

(30) Foreign Application Priority Data
Jul. 16, 2021 (GB) 2110296.7
Jul. 16, 2021 (GB) 2110297.5

(51) Int. Cl. C09D 179/02; A01N 33/04; A01N 33/12; A01N 47/28; A01N 47/40; A01P 1/00; A61K 31/785; A61P 31/02; C09D 5/14

(52) U.S. Cl. CPC C09D 179/02; A01N 33/04; A01N 33/12; A01N 47/28; A01N 47/40; A01P 1/00; A61K 31/785; A61P 31/02; C09D 5/14

(57) ABSTRACT

The present disclosure relates to coatings, formulations and coating compositions, methods for using the coatings and coating compositions, and methods for applying the coatings to substrates or articles. The coatings disclosed herein are long-lasting, and are suitable for application to a wide variety of substrates or articles, which may include substrates or articles formed from natural and/or artificial materials, including plastic, metal, textiles and/or other artificial materials, and also to living substrates.

### FIELD OF THE DISCLOSURE

The present disclosure relates to anti-microbial coatings, anti-microbial formulations and coating compositions, methods for using the anti-microbial coatings and coating compositions, and methods for applying anti-microbial coatings to substrates or articles. The coatings disclosed herein are effective to provide long-lasting anti-microbial surface action and infection prevention. The coating compositions may be suitable for application to a wide variety of substrates or articles, which may include substrates or articles formed from natural and/or artificial materials, including plastic, metal, textiles and/or other artificial materials; and may be suitable for use in the healthcare sector, particularly in clinical settings such as in hospital, for example, to impart anti-microbial activity, including biocidal and/or biostatic activity, to porous and/or non-porous surfaces, including surfaces of medical devices, tools and equipment, and/or meshes and fabrics, for example masks and dressings; thereby reducing the microbial burden on these surfaces and inhibiting the spread of infection, and preventing biofilm formation. The coatings may be deployed to medical devices, including implantable devices, personal protective equipment and surfaces in healthcare, veterinary, dentistry, industrial, military, transport, public, commercial and private settings. Coating compositions disclosed herein may also be suitable for application to body parts, particularly the skin of living human and animal subjects, and may be useful as an effective and durable skin sanitiser, for personal protection and for reducing onward spread of infection, including by way of a “touch clean” effect as herein disclosed.

### BACKGROUND

Pathogenic micro-organisms, including bacteria (gram positive and gram negative), viruses (enveloped and non-enveloped), yeast and fungi, can cause serious, debilitating and sometimes life-threatening disease. Escherichia coli is responsible for many common bacterial infections, including cholecystitis, bacteremia, cholangitis, urinary tract infections, and diarrhoea, and is also implicated in other clinical infections including neonatal meningitis and pneumonia. Gastroenteritis caused by norovirus infection results in an estimated 70,000 deaths among children aged under 5 years annually worldwide, and is estimated to give rise to a global total of US$4.2 billion in direct health system costs and $60.3 billion in societal costs each year (Bartsch et al, Global Economic Burden of Norovirus Gastroenteritis, PLoS One: 2016; 11(4)). Over 70 million cases of COVID-19, caused by the SARS-CoV-2 virus, have been recorded globally in 2020, and the ongoing pandemic is estimated to have caused over 4 million deaths worldwide. These deaths have included both patients and healthcare workers. In January 2021, the WHO Pan America Regional Office reported that by that time 570,000 healthcare workers had been affected and 2,500 had died due to COVD-19, as reported in Erdem et al Int J Infect Dis 2021 Jan: 102:239-241.

Health care associated infections (HCAIs), also referred to as hospital-associated infections or nosocomial infections, are a major concern and significant clinical burden, often with high morbidity and mortality; as discussed, for example, in Khan et al. Asian Pac J Trop Biomed 2017; 7(5); 478-482; in Haque et al, Infec Drug Res 2018:11 2321-2333; and in Aljamali et al, IJAER 2020(20) 7-20. The US Center for Disease Control and Prevention estimates that 1.7 million hospitalized patients annually acquire HCAIs while being treated for other health issues, and that more than 98,000 patients (one in 17) die due to these infections. These are infections which are acquired or which spread in a healthcare setting, and are categorised as infections which first appear 48 hours or more after hospitalisation or within 30 days after receiving health care. HCAIs include catheter-associated urinary tract infections, gastrointestinal infections, central line-associated bloodstream infections, surgical site infections, ventilator-associated pneumonia, hospital-acquired pneumonia, and MRSA (methicillin-resistant S. aureus) and Clostridium difficile infections. Other nosocomial pathogens include viral pathogens, such as influenza and SARS-CoV-2; and fungal pathogens, such as C. albicans. Such infections are particularly problematic, not least because of the ease with which they can spread in a clinical setting, and the vulnerability of many patients who are exposed to transmission of infection, owing to illness and/or immunocompromised status. Combatting the transmission of HCAIs in health care settings is therefore a significant clinical priority.

Contact transmission plays a part in the spread of many infections and infectious diseases. This includes both direct transmission, where micro-organisms are spread directly from an infected individual to another individual by close contact, including direct exposure to bodily fluids by, for example, breathing or sneezing; and indirect transmission, where micro-organisms that are present on a contaminated surface, such as the surface of a medical device or item of equipment in a health care setting, are picked up by contact. Whilst many micro-organisms, including SARS-CoV-2 and the influenza virus, are incapable of causing disease whilst they remain on the skin, any contact between contaminated skin and mucous membranes, for example of the eyes, mouth, or nose, may provide a pathway for the pathogen to enter the body. Infection may also spread in the course of intimate bodily contact by contaminated medical devices, such as implants, catheters, endoscopes and the like. In a busy healthcare environment, sanitised surfaces, including personal protective equipment (PPE), become constantly re-contaminated. Continuous repeated sanitisation of frequently touched or contaminated surfaces is therefore essential in order to prevent cross-contamination and infection spread as health professionals move from patient to patient.

Biofilm formation also plays a notable part in the spread of health care associated infections. Implantable medical devices are prone to suffer from bacterial colonisation, which leads to device related infections resulting in morbidity with significant mortality for device related infections (Li et al. Coatings 2021, 11, 294). The adhesion of bacteria onto an implantable medical device or to proteins already deposited on the device provides a suitable breeding ground for bacterial colonization, which can lead to biofilm formation. Device associated biofilms are a primary cause of hospital-acquired (nosocomial) infections. According to Li et al. (2021) approximately 2 million nosocomial infection cases occurred in the United States each year at the beginning of the 21st century, of which 50%-70% of nosocomial infections were related to indwelling medical devices. Combatting biofilm formation on medical devices in health care settings is therefore also a significant clinical priority.

There is, therefore, an ongoing need for effective surface active anti-microbial compositions to assist in reducing or preventing contact transmission of micro-organisms and the spread of infection and infectious disease. In particular, there is a need for anti-microbial compositions which are suitable and effective for use as disinfectants or disinfectant coatings on inanimate surfaces, to reduce the load and/or to inactivate and/or prevent the life or growth of micro-organisms on an inanimate surface, and/or to prevent the formation of and/or to disrupt and/or remove a surface biofilm. This can assist in limiting indirect (object to person) contact transmission via contaminated surfaces, and/or improving the efficacy of personal protective equipment (PPE) by reducing or eliminating microbial contamination during use; thereby also limiting direct (person to person) transmission during close contact. There is a need for anti-microbial compositions which are suitable and effective for use on the body as antiseptics or skin sanitisers, to reduce the load and/or to inactivate and/or prevent the life or growth of micro-organisms present on the surface of the body, thereby providing personal protection against infection and/or limiting onward transmission of infection, including both direct (person to person or animal/human to human/animal) and indirect (object to person) contact transmission.

There is an ongoing need for anti-microbial compositions with long-lasting effect, which can be applied as a disinfectant or antiseptic, including a disinfectant or antiseptic surface coating, to substrates including body parts and skin, and which may be expected to be active against micro-organisms over an extended period of time following application, and/or may be able to withstand harsh conditions, including wet and dry abrasion and water washing, and/or may be capable of being removed from skin by washing with soap and warm water.

The provision of anti-microbial compositions and coatings, showing anti-microbial efficacy, stability, and other favourable characteristics, especially longevity without reduction in efficacy, and enhanced antiviral activity, is a desirable objective in the art.

### SUMMARY OF THE DISCLOSURE

The present disclosure provides improved anti-microbial compositions and coatings, which may be suitable for application to a wide variety of substrates, including inert (that is, non-living) surfaces which may be natural and/or artificial, porous and/or non-porous, biodegradable and/or non-biodegradable; and/or including living surfaces such as the skin and body parts of living humans and animals. The compositions and coatings may be effective to provide anti-microbial action, including biocidal and/or biostatic action, against a range of micro-organisms including bacteria, viruses, yeast and fungi; thereby assisting in the prevention of infection and in reducing the spread and/or acquisition of infectious pathogens and/or infectious disease. The compositions and coatings may be effective to prevent the formation of and/or to disrupt and/or remove a surface biofilm on a substrate or article.

According to one aspect of the present disclosure, there is provided an anti-microbial coating which comprises an alkyl urea polyalkylene imine polymer. Optionally, the coating may further comprise an anionic component such as an anionic polymer. Optionally, the anti-microbial coating may further comprise one or more additional cationic polymers, such as a polyalkylene imine polymer, such as an unsubstituted polyalkylene imine polymer. Additionally or alternatively, the anti-microbial coating may further comprise a guanidine compound.

As described in more detail hereinbelow, the present inventors have found that such surface coatings can be surprisingly, strongly and durably effective against a range of micro-organisms including bacteria (gram positive and gram negative), viruses (enveloped and non-enveloped), yeast and fungi, providing a biostatic and/or biocidal effect which is capable of inhibiting biofilm formation or disrupting existing biofilms. The coatings can be deployed as a disinfectant layer for reduction of micro-organisms on inert (that is, non-living) surfaces, including but not limited to plastics, metal, textiles (natural and synthetic woven and non-woven), glass, ceramics, wood, rubber, fibres and other artificial and natural substrates, including biodegradable and non-biodegradable substrates. In addition, the coatings may be suitable for use in the healthcare, including dentistry, or veterinary sectors, including in primary, secondary and/or tertiary healthcare settings; for example, for application to medical devices or equipment, including implantable devices, and personal protective equipment. The coatings may also be effective and useful in transport settings, including industrial transport and public transport, for example for coating contact surfaces in trains or aeroplanes. The coatings may be effective and useful in public and/or commercial settings, such as in schools, bars, restaurants, hotels, gyms, spas, stadiums, offices, and any other settings where individuals may come into close contact or where infection may spread. The coatings may be effective and useful in private settings, including in the home. The coatings may be effective on porous and/or non-porous substrates, including nets, gauzes, filters and fabrics. The coatings may be used on any surfaces which are susceptible to microbial contamination, such as walls, counters, handles, tables, doors, floors, curtains, handrails, chairs or beds, or on consumer items such as toys or phones. The coatings may also be effective to reduce micro-organisms on other surfaces on contact, including on living and/or non-living surfaces, natural and/or non-natural surfaces; demonstrating a “touch-clean” effect as described herein. The coatings may also be deployed as an antiseptic or sanitiser for reduction of micro-organisms on living human or animal bodies.

According to another aspect of the disclosure, there is provided a substrate or article, such as a medical device or implant, that is coated with the anti-microbial coating of this disclosure; which substrate or article is not a part of a living human or animal body. Substrates and articles coated according to this disclosure include medical devices and implants used in the healthcare (including dentistry) and veterinary sectors; including catheters, endoscopes, cardiac implants such as stents, heart valves and biodegradable, non-biodegradable, natural and/or synthetic scaffolds and grafts, bone and joint implants, surgical tools and other types of diagnostic, surgical and therapeutic equipment.

According to another aspect of the present disclosure, there is provided a liquid coating composition suitable for forming an anti-microbial coating in accordance with this disclosure, which liquid coating composition comprises an alkyl urea polyalkylene imine. Optionally, the composition may comprise a blend of an alkyl urea polyalkylene imine polymer and an anionic component such as an anionic polymer. Optionally, the composition or the blend may comprise a blend of an alkyl urea polyalkylene imine and one or more additional cationic polymers, such as a polyalkylene imine polymer, for example, an unsubstituted polyalkylene imine polymer, including an unsubstituted polyethylene imine polymer or an unsubstituted polypropylene imine polymer. This includes embodiments in which the composition comprises a blend of an alkyl urea polyalkylene imine, an anionic component and one or more additional cationic polymers. Additionally or alternatively, the composition or blend may comprise a blend of an alkyl urea polyalkylene imine and a guanidine compound. This includes embodiments in which the composition comprises a blend of an alkyl urea polyalkylene imine, an anionic component and a guanidine compound; embodiments in which the composition comprises a blend of an alkyl urea polyalkylene imine, one or more additional cationic polymers and a guanidine compound; and embodiments in which the composition comprises a blend of an alkyl urea polyalkylene imine, an anionic component, one or more additional cationic polymers, and a guanidine compound.

According to yet another aspect, the present disclosure provides methods for coating a substrate or article with an anti-microbial coating in accordance with this disclosure. Optionally, the substrate or article may not be a part of a living human or animal body. The substrate or article may be formed from porous and/or non-porous materials.

Optionally, the method for coating a substrate or article may comprise applying to the substrate or article a liquid coating composition in accordance with this disclosure. The step of applying the liquid coating composition to the substrate or article may optionally comprise (i) incubating the substrate or article in the liquid coating composition, and/or (ii) immersing the substrate or article in the liquid coating composition, and/or (iii) washing the substrate or article with the liquid coating composition, and/or (iv) dipping the substrate or article into the liquid coating composition either once or more than once, and/or (v) causing the liquid coating composition to flow over the substrate or article, and/or (vi) spraying the liquid coating composition onto the substrate or article, and/or (vii) painting the liquid coating composition onto the substrate or article, and/or (viii) wiping the liquid coating composition onto the substrate or article, and/or (ix) brushing the liquid coating composition onto the substrate or article, and/or (x) padding the liquid coating composition onto the substrate or article, and/or (xi) rolling the liquid coating composition onto the substrate or article, and/or (xii) applying the liquid coating composition to the substrate or article by physical deposition, and/or (xiii) applying the liquid coating composition to the substrate or article by electrophoretic deposition; or any combination or sequence of these application methods, which may be performed or repeated once or more than once. Optionally, the method may further comprise drying the coated substrate or article or allowing the coated substrate or article to dry.

Alternatively, the method for coating a substrate or article with an anti-microbial coating according to this disclosure may comprise the sequential steps of:
(a) applying a first liquid composition comprising one or more of an alkyl urea polyalkylene imine polymer as herein defined, an anionic component such as an anionic polymer as herein defined, an additional cationic polymer(s) as herein defined, and a guanidine compound as herein defined to the substrate or article one or more times to form a first layer; followed by
(b) applying a second liquid composition different from the first liquid composition and comprising one or more of an alkyl urea polyalkylene imine polymer as herein defined, an anionic component such as an anionic polymer as herein defined, an additional cationic polymer(s) as herein defined, and a guanidine compound as herein defined to the substrate or article one or more times to form a second layer; followed by
(c) optionally repeating step (a) and/or step (b);
such as to form an anti-microbial surface coating comprising an alkyl urea polyalkylene imine polymer in accordance with this disclosure.

The method may, optionally, include a subsequent step (d) of applying a third and, optionally, a subsequent liquid composition different from the first and/or second liquid compositions and comprising one or more of an alkyl urea polyalkylene imine polymer as herein defined, an anionic component such as an anionic polymer as herein defined, an additional cationic polymer(s) as herein defined and a guanidine compound as herein defined to the substrate or article one or more times to form a third and, optionally, subsequent layer.

The present disclosure further comprises substrates and articles which are coated with an anti-microbial coating in accordance with the disclosure, which substrates or articles are not part of a living human or animal body. Said substrates or articles may be inert (non-living), and/or may be formed from natural and/or artificial materials, including but not limited to natural and synthetic polymers/plastics, metal, glass, silica/silicone, ceramics, marble/stone, composites, wood, rubber, fabrics and textiles such as natural and/or synthetic fibres. Said substrates or articles may be formed from porous materials. Said substrates or articles may be formed from non-porous materials. Said substrates or articles may be partly porous and partly non-porous. Such substrates and articles may include medical devices or equipment. Such substrates and articles may include personal protective equipment, and/or equipment which is used to clean other surfaces, such as cloths, wipes or brushes.

The disclosure further provides a method for preventing or reducing the growth or spread or the load or quantity of one or more micro-organisms on a substrate or article, and/or for inactivating one or more micro-organisms on a substrate or article, and/or for preventing the formation of and/or disrupting and/or removing a surface biofilm on a substrate or article, comprising the step of applying a coating to the substrate or article according to the present disclosure. The substrate may be an inert (non-living) substrate. The substrate may be a body part of a living human or animal, such as the skin, for example the hands or face or feet of a human.

The disclosure further embraces the use of a liquid coating composition in accordance with the disclosure for preventing or reducing the growth or spread or the quantity of one or more micro-organisms on a substrate, and/or for inactivating one or more micro-organisms on a substrate, and/or for preventing the formation of and/or disrupting and/or removing a surface biofilm on a substrate or article, whereby the liquid coating composition is applied to the substrate in accordance with the present disclosure. The substrate may be an inert (non-living) substrate, or may be a body part of a living human or animal, such as the skin, such as the hands or face or feet of a human.

The disclosure further provides a liquid coating composition in accordance with the disclosure, for use in a method of preventing or reducing the growth or spread or the quantity of one or more micro-organisms on a body part of a living human or animal, and/or for inactivating one or more micro-organisms on a body part of a living human or animal, which method comprises applying the liquid coating composition to the body part; optionally, by washing or rinsing the body part with the liquid coating composition and/or by spraying, rubbing, padding, rolling, depositing and/or brushing the liquid coating composition onto the body part. The body part may, for example, be the skin, such as the hands or face or feet of a human.

The disclosure further provides a method for preventing or reducing the growth or spread or the load or quantity of one or more micro-organisms on a surface, and/or for inactivating one or more micro-organisms on a surface, and/or for preventing the formation of and/or disrupting and/or removing a surface biofilm on a substrate or article, comprising the step of contacting the surface with a substrate or article which comprises a coating in accordance with this disclosure and/or which has been coated according to this disclosure. The coated substrate or article may be an item of cleaning equipment, such as a cloth or sponge. The coated substrate or article may be an item of personal protective equipment, such as gloves or a mask or a face shield or medical scrubs or surgical gowns or eye protection. The coated substrate or article may be a body part of a living human, such as the skin, for example the hands or face or feet of a human. The surface may be any surface that is or may be or may become contaminated with micro-organisms, including surfaces in healthcare settings, public settings, or private settings, including primary, secondary and tertiary healthcare settings; and surfaces of medical devices and equipment; and parts of the human or animal body, including the skin.

The disclosure further provides an alkyl urea polyalkylene imine polymer for use in preventing or reducing the growth or spread or the load or quantity of one or more micro-organisms, including bacteria, viruses, fungi and/or yeast. The disclosure provides for the use of an alkyl urea polyalkylene imine polymer in preventing or reducing the growth or spread or the load or quantity of one or more micro-organisms, including bacteria, viruses, fungi, and/or yeast.

### BRIEF DESCRIPTION OF THE FIGURES

Anti-microbial coatings of the disclosure including a stain are shown in FIGS. 1-6 applied to the fingers and/or hands of a volunteer as follows:

FIG. 1 shows a dyed coated index finger of volunteer before water wash, where the dye (indicating the presence of the coating) is clearly visible and uniformly distributed;

FIG. 2 shows dyed fingers of volunteer: (a) Uncoated finger after water wash, showing minimal adherence of dye (b) Coated finger after water wash and abrasion, where the dye is clearly visible and uniformly distributed;

FIG. 3 shows a dyed coated index finger submerged in artificial sweat, after water wash and abrasion; where the dye is clearly visible and uniformly distributed;

FIG. 4 shows a dyed coated hand of volunteer left in glove for 18 hours before water wash, where the dye is clearly visible and uniformly distributed;

FIG. 5 shows a dyed coated hand of volunteer left in glove for 18 hours after water wash, where the dye is clearly visible and uniformly distributed;

FIG. 6 shows a coated hand of volunteer left in glove for 18 hours after washing with soap and warm water; showing minimal presence of dye and therefore of coating;

FIG. 7 shows a graph showing the measured dynamic CoF values of coatings with and without alkyl urea polyalkylene imine polymer, measured over 20 cycles, where the coatings are applied using a layered coating method on a TPU strip;

FIG. 8 shows a graph showing the measured dynamic CoF values of coatings with and without alkyl urea polyalkylene imine polymer, measured over 20 cycles, where the coatings are applied as a blended formulation on a TPU strip;

FIG. 9 illustrates the durability and resistance to abrasion of coatings of composition D1 when applied to surgical masks and TPU; by comparing the dye uptake of a coated substrate after abrasion (visible and uniform distribution of dye) with that of an uncoated substrate (minimal adherence of dye);

FIG. 10 illustrates the non-leaching character of coatings of composition D1 when applied to TPU; by comparing the zone of inhibition in E. coli created by a coated TPU strip vs a positive control catheter coated with chlorhexidine/silver. This Figure shows no zone of inhibition on the coated TPU and a zone of inhibition on the coated catheter (chlorhexidine/silver) demonstrating no leaching and leaching, respectively.

### DETAILED DESCRIPTION

The disclosure provides an anti-microbial coating which comprises an alkyl urea polyalkylene imine polymer. Optionally, the coating may further comprise an anionic component such as an anionic polymer. Optionally, the anti-microbial coating may further comprise one or more additional cationic polymers that are distinct from the alkyl urea polyalkylene imine polymer, such as a polyalkylene imine polymer, such as an unsubstituted polyalkylene imine polymer. Additionally or alternatively, the anti-microbial coating may further comprise a guanidine compound. The anti-microbial coating may further comprise one or more further active agents, excipients and/or additives, including additional anti-microbial agents such as quaternary ammonium compounds and/or binders, as herein disclosed.

Polyalkylene imine polymers are well known in the art. They are straight-chain or branched-chain polymers having a backbone formed from repeating units of an amine group and an alkyl spacer group, which may, for example, be a C₁₋₁₀ alkyl spacer group. Polyethylene imine polymers, for example, have a backbone formed from repeating units of an amine group and an ethylene spacer group, as shown in Structural Formula 1 below:

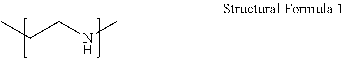

Alkyl urea polyalkylene imine polymers, as defined and utilised herein, are N-derivatised polyalkylene imine polymers, which have at least one alkyl group that is attached to the polyalkylene imine polymer backbone by way of at least one urea linkage that includes a nitrogen heteroatom on the polyalkylene imine polymer backbone. A urea linkage is illustrated in Structural Formula 2 below:

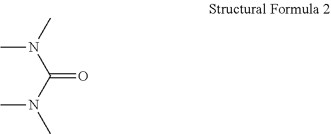

A part of an exemplary alkyl urea polyalkylene imine polymer as herein defined is illustrated in Structural Formula 3 below. This exemplary polymer is a branched-chain alkyl urea polyethylene imine polymer that includes a plurality of alkyl groups R. Each alkyl group R is attached by way of a urea linkage that includes a nitrogen heteroatom on the polyethylene imine polymer backbone. Each alkyl group R is attached at one point to the polyethylene imine polymer backbone, thereby forming a pendant alkyl urea side group:

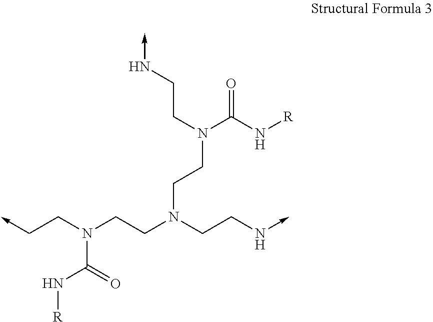

A part of a further exemplary alkyl urea polyalkylene imine polymer as herein defined is illustrated in Structural Formula 4 below. This exemplary polymer is a straight-chain alkyl urea polyethylene imine polymer that includes a plurality of alkyl groups R. Here, each alkyl group R is attached by way of two urea linkages, each including a nitrogen heteroatom on the polyethylene imine polymer backbone, so as to form cross-linking alkyl urea groups, which cross-link the polyethylene imine polymer backbone:

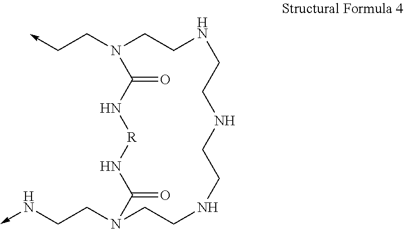

Polyethylene imines have been described in the art as antimicrobial agents with selective activity (Gibney et al, Macromol Biosci 2012 12(9) 1279-1289). It has also been suggested in the art that glass slides painted with the hydrophobic long-chained polycation N-N-dodecyl,methyl-polyethylene imine (N,N-dodecyl,methyl-PEI) is highly effective against waterborne influenza A viruses (Haldar et al, Biotechnol Lett 2008 30:475-479). The present inventors have, however, identified that alkyl urea polyalkylene imine polymers, bearing alkyl substituents linked to the polyalkylene imine polymer backbone via a urea linkage as herein disclosed, are more hydrophilic in character than the corresponding alkylated polyalkylene imine polymer, owing to the presence of the hydrophilic urea functionality. The present inventors have identified and demonstrate experimentally herein that coatings comprising alkyl urea polyalkylene imine polymers display particularly effective anti-microbial properties, whilst also showing improved surface adherence, stability, durability and/or mechanical characteristics, and/or displaying surprising functional properties as disclosed herein.

The alkyl groups in an alkyl urea polyalkylene imine according to this disclosure may comprise or consist of a straight chain or branched free alkyl chain, such as an alkyl chain terminating in one or more —CH₃ groups; and/or may comprise or consist of a cyclic alkyl group which is cyclised with itself, that is, a cycloalkyl group.

In particular, each alkyl group may comprise a straight or branched alkyl chain and/or a cycloalkyl group. Typically, each alkyl group may comprise no more than 15 carbon atoms, favourably no more than 10 carbon atoms, or no more than 6 carbon atoms, or fewer than 6 carbon atoms, in a branched or linear saturated chain. For example, each alkyl group may comprise straight or branched chain methyl, ethyl, propyl, butyl, pentyl, hexyl, heptyl, octyl, nonyl or decyl; and/or cyclobutyl, cyclopentyl, cyclohexyl, cycloheptyl, cyclooctyl, cyclononyl or cyclodecyl.

In some favoured embodiments, each alkyl group may be selected from methyl, ethyl, propyl, butyl or pentyl, and each alkyl group may be attached to the polyalkylene imine polymer backbone by way of a single urea linkage, so as to form an alkyl urea side group that is pendant to the polyalkylene imine polymer backbone. In such embodiments, the alkyl urea polyalkylene imine polymer may be a polyalkylene imine having one or more R—NH—C(O)— groups attached to a nitrogen heteroatom in the polyalkylene imine chain where R is an alkyl group as herein defined. In other favoured embodiments, each alkyl group may be selected from propyl, butyl, pentyl, hexyl, heptyl, octyl, nonyl or decyl, and each alkyl group may be attached to the polyalkylene imine polymer backbone by way of two or more urea linkages, so as to form a cross-linking alkyl urea group. In such embodiments, the alkyl urea polyalkylene imine polymer may be a polyalkylene imine having a —C(O)—NH—(CR′₂)ₙ—NH—C(O)— linkage between two nitrogen heteroatoms in the polyalkylene imine chain where each R′ is hydrogen or substituent such as alkyl or halide on an alkylene chain. Some embodiments of the present disclosure may comprise both pendant alkyl urea groups and cross-linking alkyl urea groups, as herein defined.

In some preferred embodiments, the alkyl urea polyalkylene imine may be a polyethylene imine which comprises one or more pendant ethyl urea, propyl urea, butyl urea or pentyl urea groups. In particular, the alkyl urea polyalkylene imine may be a butyl urea polyethylene imine. Optionally, the alkyl urea polyalkylene imine may be a polyalkylene imine which comprises one or more cross-linking alkyl groups each comprising an alkyl group attached to the polyalkylene imine by way of two or more urea linkages; where the alkyl group may comprise 1-10 carbon atoms, favourably 3-7 carbon atoms. In particular, the alkyl urea polyalkylene imine may be a hexamethylene diurea polyethylene imine. Each alkyl group of the alkyl urea polyalkylene imine polymer preferably comprises no more than 10 carbon atoms, not including the sp² carbons of the urea linkage(s), preferably no more than 8 carbon atoms, not including the sp² carbons of the urea linkage(s).

The alkyl urea polyalkylene imine may, for example, be obtained by reacting a polyalkylene imine with an isocyanate or diisocyanate to form a carbamide/urea derivative (see Jager et al. Chem. Soc. Rev., 2012, 41, 4755-4767 which shows this reaction schematically on page 4760). Primary & secondary amines such as polyalkylene imines react with isocyanates to give substituted ureas in the reaction R—N═C═O+R′R″NH→R—NH—(C═O)—NR′R″, where in the case of a polyalkylene imine R′ and R″ are both alkylene groups in the polyalkylene imine polymer backbone.

An alkyl urea polyalkylene imine according to the present disclosure may, for example, be an alkyl urea polyethylene imine polymer and/or an alkyl urea polypropylene imine polymer. The alkyl urea polyalkylene imine may be a branched or straight-chain polyalkylene imine polymer; and may in particular be a highly branched polyalkylene imine polymer. The alkyl urea polyalkylene imine polymer may, optionally, be further substituted with one or more inert substituents, such as halogen substituents. The alkyl urea polyalkylene imine polymer may, for example, have a molecular weight of up to 2 MDa, or up to 1 MDa, or up to 750 kDa, or up to 500 kDa, or up to 250 kDa, or up to 100 kDa. The alkyl urea polyalkylene imine polymer may have a molecular weight of at least 500 Da, or at least 800 Da, or at least 1 kDa, or at least 2 kDa, or at least 5 kDa, or at least 10 kDa, or at least 25 kDa. The alkyl urea polyalkylene imine polymer may have a molecular weight in the range 800 Da-2 MDa or 1 kDa-1 MDa; or 1 kDa-500 kDa, or 1 kDa-100 kDa, or 10 kDa-100 kDa.

As reported hereinbelow, and as demonstrated in the experimental examples, the present inventors have shown that the compositions and coatings according to the present disclosure have strong and durable anti-microbial effect against a wide range of different micro-organisms. As illustrated in the examples, the compositions and coatings may have long-term antimicrobial activity (antibacterial, antiviral, yeasticidal, and/or fungicidal; biocidal and/or biostatic) when applied to a range of non-porous and porous surfaces, as well as skin with a long-term efficacy (180-365 days for surfaces and 48 hours for skin). More specifically, disinfectant compositions within the present disclosure are capable, as shown, of producing stable, durable and lubricious coatings with excellent mechanical characteristics and high levels of adhesion and durability when applied to non-porous surfaces, such as TPU. The coated surfaces are capable, as shown, of withstanding harsh conditions, such as dry and wet abrasion (water and chemical resistance) on non-porous surfaces, without significant deterioration in quality or performance. More specifically, disinfectant compositions within the present disclosure are capable, as shown, of producing stable, and durable coatings when applied to porous surfaces such as masks. Furthermore, the coatings are shown in the examples to be non-leaching, which is a significant advantage, not least for environmental reasons.

Compositions within the present disclosure are capable of producing anti-microbial coatings which are biocompatible, and effective and useful as sanitisers on skin, showing water-resistant properties which improve their durability, including to abrasion, when used on skin, but are nevertheless capable of being removed from the skin by washing with soap and warm water. This is an important advantage, particularly for skin sanitiser compositions. This property is also demonstrated in the experimental examples. These compositions also show excellent levels of anti-microbial activity, as demonstrated in the examples.

As described and exemplified herein, the disclosed compositions are capable of yielding anti-microbial coatings which may be effective to reduce microbes and pathogens on other surfaces on contact, by way of a “touch-clean” effect. This is a surprising attribute which has not previously been described in the art.

The coatings of the present disclosure comprise an alkyl urea polyalkylene imine polymer. Optionally, the coatings may further comprise an anionic component such as an anionic polymer. In some embodiments, the anionic polymer may be an anionic polyelectrolyte. This allows electrostatic interactions between the components of the coating, specifically between the anionic component and the cationic components including the alkyl urea polyalkylene imine, which may help to improve the stability and performance of the coating. Suitable anionic components, polymers and polyelectrolytes are known in the art. The anionic polymer may be an anionic glycosaminoglycan or polysaccharide, such as dextran sulfate; or a polycarboxylic acid polymer, such as a polyacrylic acid polymer or a salt thereof. Preferably, the anionic polymer may be a polyacrylic acid polymer.

A coating according to the present disclosure may, optionally, comprise one or more additional cationic polymers distinct from the alkyl urea polyalkylene imine polymer. The or each additional cationic polymer may be any positively charged polymer, such as a polyamine or polyamidoamine polymer. Examples of cationic polymers include cationic peptides and their derivatives (e.g., polylysine, polyornithine), linear or branched synthetic polymers (e.g., hexadimethrine bromide (polybrene), or polyalkylene imines such as polyethyleneimine), polysaccharide-based delivery molecules (e.g., cyclodextrin, chitosan) and natural polymers (e.g., histone, collagen). The additional cationic polymer(s) may be or may include a polydiallyldialkylammonium salt, a poly(acrylamide-co-diallylalkyl ammonium halide), an acryloxyalkyltrialkylammonium salt such as acryloxyethyltrimethylammonium halide or methacryloxyethyltrimethylammonium halide, a vinylphenalkyltrialkylammonium salt such as vinylbenzyltrimethylammonium halide, an acrylamidoalkyltrialkylammonium salt such as 3-acrylamido-3-methylbutyltrimethylammonium halide, and/or a poly(acrylamide-co-diallyldialkylammonium salt) such as poly(acrylamide-co-diallyldimethylammonium chloride). The additional cationic polymer(s) may be or may include a polyalkylene imine polymer, particularly a polyalkylene imine polymer which is not an alkyl urea polyalkylene imine polymer; for example, an unsubstituted polyalkylene imine polymer, or an alkylated polyalkylene imine polymer, for example a polyalkylene imine polymer which is substituted with C₁-C₈ straight chain, branched, or cyclic alkyl. The additional cationic polymer(s) may suitably be or may include an unsubstituted or an alkyl-substituted polyethylene imine polymer or polypropylene imine polymer. Suitably, the additional cationic polymer(s) may be or may include an unsubstituted polyethylene imine polymer. The additional cationic polymer(s) may be or may include a polyalkylene imine polymer having a molecular weight of up to 2 MDa, or up to 1 MDa, or up to 750 kDa, or up to 500 kDa, or up to 250 kDa, or up to 100 kDa. The polyalkylene imine polymer may have a molecular weight of at least 500 Da, or at least 800 Da, or at least 1 kDa, or at least 2 kDa, or at least 5 kDa, or at least 10 kDa, or at least 25 kDa. The polyalkylene imine polymer may have a molecular weight in the range 800 Da-2 MDa or 1 kDa-1 MDa; or 1 kDa −500 kDa, or 1 kDa-100 kDa, or 10 kDa-100 kDa.

In some preferred embodiments, the coating or composition comprises butyl urea polyethylene imine polymer and/or hexamethylene diurea polyethylene imine polymer, alone or in combination with an unsubstituted or substituted polyethylene imine polymer, especially unsubstituted polyethylene imine polymer. Optionally, the coating or composition may further comprise an anionic component such as an anionic polymer as herein disclosed; for example, a polyacrylic acid polymer.

A coating according to the present disclosure may, additionally or alternatively, further comprise a guanidine compound. This may be particularly favourable for coatings on inert (non-living) substrates. Guanidine compounds and polymers have been identified in the art as promising anti-microbial agents. Guanidine compounds include a strongly cationic signature guanide group:

—N(R₁)—C(═NR₂)—N(R₃)—

where R₁, R₂, and R₃ may be H, alkyl, or other substituents.

A well-known example is polyhexamethylene guanidine, which is used as a biocidal disinfectant:

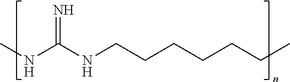

Another example is polyhexanide, or PHMB:

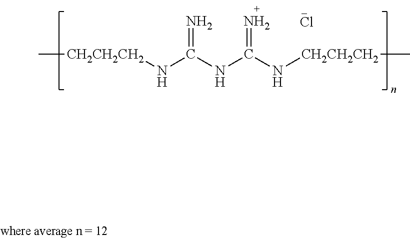

The guanidine compound according to the present disclosure may accordingly be any compound which comprises one or more guanide groups:

—N(R₁)—C(═NR₂)—N(R₃)—

where each of R₁, R₂ and R₃ may, for example, be H or alkyl, or another substituent.

One example of a guanide group is a biguanide group:

—N(R₁)—C(═NR₂)—N(R₃)—C(═NR₄)—N(R₅)—

where each of R₁, R₂, R₃, R₄ and R₅ may, for example, be H or alkyl.

In some embodiments, the guanidine compound may comprise one or more pairs of guanide groups. For example, the guanidine compound may comprise one or more bisbiguanide groups.

The guanidine compound may, for example, comprise one or more bis-biguanide groups, such as chlorhexidine groups:

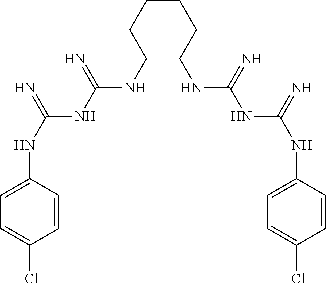 [SMILES: N=C(NCCCCCCNC(=N)NC(=N)Nc1ccc(Cl)cc1)NC(=N)Nc1ccc(Cl)cc1]

The guanidine compound may comprise one or more alexidine groups:

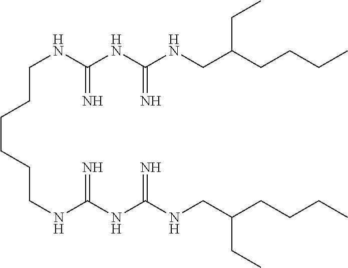 [SMILES: CCCCC(CC)CNC(=N)NC(=N)NCCCCCCNC(=N)NC(=N)NCC(CC)CCCC]

The guanidine compound may comprise one or more polyguanide segments, each comprising a plurality of guanide groups. Each polyguanide segment may comprise repeating units of a guanide group, or of a guanide group and a linking group such as an alkyl group, particularly a C₁₋₁₀ alkyl group.

In particular, each polyguanide segment may comprise one or more poly(hexamethylene) guanide segments:

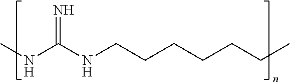

Each polyguanide segment may also or alternatively comprise one or more poly(hexamethylene) biguanide (PHMB) segments, as illustrated below or above:

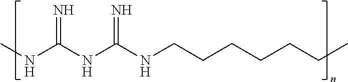

The guanidine compound may comprise a polymer having a polymer backbone which bears one or more pendant guanide groups, such as one or more biguanide groups and/or one or more bisbiguanide groups and/or one or more polyguanide segments. Pendant groups include groups that are attached to the polymer backbone. Such attachment may be achieved by copolymerising moieties (suitable species such as monomers, oligomers and the like) to yield longer chain polymer structures with pendant groups directly, or in stages, such as by initially forming one polymer, which may itself be a copolymer, from suitable species, and attaching pendant functional groups subsequently.

In a guanidine compound according to the present disclosure, some or all of the guanide group or groups may be covalently bound to the polymer backbone directly or by way of an attachment group. In some embodiments, the attachment group may comprise an alkyl group, a polyethylene oxide group, an amine group, an ether group, or a combination thereof.

The polymer may comprise a vinyl polymer having an alkyl polymer backbone. Alternatively, the polymer backbone may comprise suitable heteroatoms, such as sulphur, phosphorus, oxygen or nitrogen heteroatoms. Pendant groups may be bound to the polymer backbone by way of any suitable functionality, including hydroxyl (—OH), carboxyl (—COOH), anhydride (—CO—O—CO—), isocyanate (—NCO), allyl, vinyl, acrylate, methacrylate, epoxide, sulfonic (—SO₃⁻) or sulfate (—SO₄⁻) groups.

The polymer may comprise additional functional pendant groups, which may suitably include one or more pendant cross-linkable groups. The cross-linkable groups may for example comprise cross-linkable carboxylic acid groups. The polymer may comprise additional functional pendant groups with desirable functionality, such as lubricant or anti-fouling groups. The polymer may comprise pendant hydrophobic groups, such as pendant C₁₋₁₀ alkyl groups. The polymer may comprise pendant hydrophilic groups, such as pendant polyethylene glycol groups.

In some embodiments, the guanidine compound may comprise an anti-microbial guanidine polymer such as an anti-microbial polymer of the type disclosed and/or exemplified in WO 00/65915 or in WO 2014/174237. WO 00/65915 discloses infection resistant guanidine polymers which can be used to coat medical devices. These polymers include an infection resistant biguanide group, such as a polyhexanide group, pendant to the polymer backbone. The specific examples describe dissolution of the guanidine polymers in a mixture of isopropanol and tetrahydrofuran to form a coating solution. PVC or polyurethane tubing is then dip-coated in the coating solution to provide an anti-microbial coating. WO 2014/174237 also discloses anti-microbial guanidine coating polymers. These polymers also possess a biguanide (polyhexanide) pendant group. Devices coated with the disclosed guanidine polymers were tested and shown to display anti-microbial activity against P. aeruginosa, E. faecalis, E. coli and S. aureus. In the present disclosure, the level of anti-microbial activity imparted to the coating can be tuned by varying the amount of anti-microbial polymer that is included in the coating.

In some preferred embodiments, the disclosure provides an anti-microbial coating as defined in any of the following numbered statements 1-14:

1. An anti-microbial coating comprising an alkyl urea polyalkylene imine, an anionic polymer and optionally a cationic polymer.

2. An anti-microbial coating according to statement 1, wherein the anionic polymer is an anionic polyelectrolye, and/or wherein the anionic polymer is an anionic glycosaminoglycan or polysaccharide, such as dextran sulfate; or a polycarboxylic acid polymer, such as a polyacrylic acid polymer.

3. An anti-microbial coating according to statement 1 or statement 2, wherein the cationic polymer is a polyalkylene imine such as a polyethylene imine or a polypropylene imine; and/or wherein the alkyl urea polyalkylene imine is an alkyl urea polyethylene imine or an alkyl urea polypropylene imine.

4. An anti-microbial coating according to any of statements 1-3, wherein the alkyl urea polyalkylene imine is a polyalkylene imine which is substituted with one or more alkyl urea groups; particularly one or more methyl, ethyl, propyl, butyl, pentyl, hexyl, heptyl, octyl, nonyl or decyl urea groups; favourably one or more ethyl, propyl, butyl, or pentyl urea groups; or wherein the alkyl urea polyalkylene imine is a polyalkylene imine which is substituted with one or more one or more alkyl groups comprising alkylene chains linked to the polyalkylene imine with two or more urea linkages; particularly one or more methylene, groups, especially 1-10 methylene groups via a urea linkage at either end of an alkylene chain, favourably 3-7 methylene groups.

5. An anti-microbial coating comprising a guanidine compound, an alkyl urea polyalkylene imine polymer, an anionic polymer and optionally a further cationic polymer.

6. An anti-microbial coating according to statement 5, wherein the alkyl urea polyalkylene imine polymer is an alkyl urea substituted polyethylene imine or an alkyl urea substituted polypropylene imine.

7. An anti-microbial coating according to statement 5 or statement 6, wherein the alkyl urea polyalkylene imine is a polyalkylene imine which is either:
substituted with one or more straight chain, branched or cyclic alkyl groups, selected urea linkage, favourably one or more ethyl, propyl, butyl, or pentyl groups; or
substituted with one or more alkyl groups comprising an alkylene chain linked to the polyalkylene imine with two or more urea linkages; especially 1-10 methylene groups via a urea linkage at either end of an alkylene chain, favourably 3-7 methylene groups.

8. An anti-microbial coating according to any of statements 5-7, wherein the guanidine compound is a compound comprising one or more guanidine or biguanidine groups, such as a bisbiguanide compound.

9. An anti-microbial coating according to any of statements 5-8, wherein the guanidine compound is a polymeric compound bearing one or more pendant guanidine or biguanidine groups, such as one or more bisbiguanide groups.

10. An anti-microbial coating according to statement 9, wherein the polymeric compound comprises a vinyl polymer which can be synthesised by polymerisation of a plurality of vinyl monomers including a plurality of vinyl monomers comprising one or more guanidine or biguanidine groups, such as one or more bisbiguanide groups.

11. An anti-microbial coating according to statement 10, wherein the plurality of vinyl monomers includes one or more cross-linkable monomers, optionally bearing cross-linkable carboxylic acid groups; and/or one or more monomers bearing hydrophobic or hydrophilic groups, such as polyethylene glycol or alkyl groups.

12. An anti-microbial coating according to any of statements 9-11, wherein the guanidine or biguanidine groups include one or more chlorhexidine groups, and/or one or more polyhexanide groups, and/or one or more alexidine groups.

13. An anti-microbial coating according to any of statements 5-12, wherein the anionic polymer is an anionic polyelectrolye, and/or wherein the anionic polymer is an anionic glycosaminoglycan or polysaccharide, such as dextran sulfate; or a polycarboxylic acid polymer, such as a polyacrylic acid polymer.

14. An anti-microbial coating according to any of statements 5-13, wherein the optional further cationic polymer is a further polyalkylene imine polymer, such as an unsubstituted polyalkylene imine.

The present disclosure further embraces a liquid coating composition suitable for forming an anti-microbial coating as disclosed herein. The liquid coating composition comprises an alkyl urea polyalkylene imine polymer in accordance with this disclosure.

Optionally, the liquid coating composition may comprise a blend of the alkyl urea polyalkylene imine polymer with an anionic component such as an anionic polymer according to this disclosure. The liquid coating composition may comprise a blend of the alkyl urea polyalkylene imine polymer with one or more additional cationic polymers as disclosed herein. In some embodiments, the liquid coating composition may comprise a blend of the alkyl urea polyalkylene imine polymer, the anionic component such as the anionic polymer, and one or more additional cationic polymers as disclosed herein.

The liquid coating composition may comprise a blend of the alkyl urea polyalkylene imine polymer with a guanidine compound as disclosed herein. Such coating compositions may be particularly suitable for application to inert (non-living) substrates. In these embodiments, the blend may also comprise an anionic component such as an anionic polymer as disclosed herein, and/or the blend may also comprise one or more additional cationic polymers as disclosed herein. In embodiments where the guanidine compound comprises a polymer bearing one or more cross-linkable groups, the liquid coating composition may suitably further include a cross-linking agent for cross-linking the polymer. Any suitable cross-linking agent may be used, such as a polyfunctional aziridine cross-linking agent, such as a polyaziridine cross-linking agent; or a polycarbodiimde cross-linking agent, such as EDC (1-Ethyl-3-(3-dimethylaminopropyl)carbodiimide) or DCC (N′,N′-dicyclohexyl carbodiimide).

The liquid coating composition may further comprise one or more solvents, carriers, active agents, excipients, binders, surfactants and/or other additives; including those described in further detail below.

The liquid coating composition may optionally be formulated in a liquid medium as a solution, suspension, dispersion, or emulsion. The liquid medium may be aqueous, alcoholic, or aqueous/alcoholic. The liquid medium may comprise an organic solvent, such as a polar organic solvent. In some embodiments, the liquid medium comprises methanol, ethanol, propanol and/or isopropanol, and/or water, and/or tetrahydrofuran. In some preferred embodiments, the liquid coating composition is formulated as a solution.

A liquid coating composition according to the present disclosure may suitably comprise at least about 0.005% w/v, or at least about 0.01% w/v, or at least about 0.02% w/v, or at least about 0.03% w/v, or at least about 0.05% w/v, or at least about 0.08% w/v, or at least about 0.10% w/v, or at least about 0.15% w/v, or at least about 0.2% w/v of said alkyl urea polyalkylene imine polymer. The liquid coating composition may typically comprise at least 0.1% w/v, for example at least 0.15% w/v, or at least 0.2% w/v, of total cationic polymers, including the alkyl urea polyalkylene imine and any additional cationic polymer(s) (where included). Thus, for compositions which do not contain additional cationic polymer(s), the composition may optionally comprise at least 0.1% w/v, for example at least 0.15% w/v, especially at least 0.2% w/v alkyl urea polyalkylene imine. Compositions comprising additional cationic polymer(s), for example a further polyalkylene imine as herein disclosed, may optionally comprise at least about 0.005% w/v, or at least about 0.01% w/v, or at least about 0.02% w/v, or at least about 0.03% w/v, or at least about 0.05% w/v, or at least about 0.08% w/v, or at least about 0.10% w/v of said alkyl urea polyalkylene imine. Such compositions may comprise a quantity of additional cationic polymer(s) such that the total content of cationic polymer in the composition, including the alkyl urea polyalkylene imine polymer and the additional cationic polymer(s), amounts to at least 0.1% w/v, or at least 0.15% w/v, or at least 0.2% w/v, as herein disclosed.

A liquid coating composition within this disclosure may suitably comprise no more than about 25% w/v, or no more than about 20% w/v, or no more than about 15% w/v, or no more than about 10% w/v, or no more than about 8% w/v, or no more than about 7% w/v, or no more than about 6.5% w/v, or no more than about 6% w/v, or no more than about 5% w/v of said alkyl urea polyalkylene imine polymer. Alternatively, particularly where the composition comprises one or more additional cationic polymers, the composition may suitably comprise no more than about 10% w/v, or no more than about 8% w/v, or no more than about 7% w/v, or no more than about 6.5% w/v, or no more than about 5% w/v, or no more than about 4% w/v, or no more than about 3% w/v, or no more than about 2% w/v, or no more than about 1% w/v, or no more than about 0.9% w/v, or no more than about 0.75% w/v, or no more than about 0.7% w/v, or no more than about 0.6% w/v, or no more than about 0.5% w/v, of said alkyl urea polyalkylene imine.

A liquid coating composition according to the present disclosure may suitably comprise at least about 0.01% w/v, or at least about 0.02% w/v, or at least about 0.04% w/v, or at least about 0.05% w/v, or at least about 0.1% w/v, or at least about 0.15% w/v, or at least about 0.2% w/v, or at least about 0.3% w/v, or at least about 0.5% w/v, of additional cationic polymer(s). The composition may suitably comprise no more than about 25% w/v, or no more than about 20% w/v, or no more than about 15% w/v, or no more than about 10% w/v, or no more than about 8% w/v or no more than about 7% w/v, or no more than about 6% w/v, or no more than about 5% w/v, or no more than about 4% w/v, of additional cationic polymer(s). In particular, the coating composition may comprise between about 0.1-10% w/v of additional cationic polymer(s); or may comprise between about 0.15-7% w/v or between 0.3-8% w/v, or between about 0.2-5% w/v, or between about 0.1-1% w/v of additional cationic polymer(s).

Compositions comprising at least about 0.01% w/v, or at least about 0.05% w/v, or at least about 0.1% w/v, or at least about 0.15% w/v, or at least about 0.2% w/v of additional cationic polymer(s), may optionally comprise no more than about 0.9% w/v, or no more than about 0.75% w/v, or no more than about 0.7% w/v, or no more than about 0.6% w/v, or no more than about 0.5% w/v, of said alkyl urea polyalkylene imine. The liquid coating composition may, for example, comprise a blend of an alkyl urea polyalkylene imine, and an additional cationic polymer(s), such as polyalkylene imine, and optionally also an anionic component such as an anionic polymer, wherein the composition comprises between about 0.02-0.9% w/v of said alkyl urea polyalkylene imine and between about 0.2-5% w/v of said additional cationic polymer(s). Such compositions may be particularly useful and applicable for use on living substrates, and may for example be useful as skin sanitisers or antiseptics.

The liquid coating composition may comprise a blend of an alkyl urea polyalkylene imine, and an additional cationic polymer(s), such as polyalkylene imine, wherein the composition comprises between about 0.01-2% w/v of said alkyl urea polyalkylene imine and between about 0.1-1% w/v of said additional cationic polymer(s); and wherein the composition optionally also comprises an anionic component such as an anionic polymer, optionally in an amount between about 0.005-0.3% w/v. Such compositions may be particularly useful and applicable for use on living substrates, and may for example be useful as skin sanitisers or antiseptics.

The liquid coating composition may comprise a blend of an alkyl urea polyalkylene imine, and an additional cationic polymer(s), such as polyalkylene imine, and optionally also an anionic component such as an anionic polymer, wherein the composition comprises between about 0.05-7% w/v of said alkyl urea polyalkylene imine and between about 0.3-8% w/v of said additional cationic polymer(s). The additional cationic polymer(s) may include a polyalkylene imine, typically an unsubstituted polyalkylene imine. The composition may optionally further include an anionic component such as an anionic polymer, optionally in an amount 0.005-0.3% w/v. The composition may optionally further include one or more binders, as disclosed herein. Such compositions may be particularly useful and applicable for use on porous and/or non-porous surfaces.

Suitably, the total cationic polymer content of the composition, including the alkyl urea polyalkylene imine and any additional cationic polymer(s) may be no more than about 25% w/v, or no more than about 23% w/v, or no more than about 20% w/v, or no more than about 18% w/v, or no more than about 15% w/v, or no more than about 12% w/v, or no more than about 10% w/v, or no more than about 8% w/v, or no more than about 7% w/v, or no more than about 6.5% w/v, or no more than about 6% w/v, or no more than about 5% w/v.

The coating composition may favourably comprise between about 0.01-70.0% w/v, or between about 0.02-5% w/v, or between about 0.03-3.5% w/v, or between about 0.1-6% w/v, or between about 0.01-2% w/v, or between about 0.05-7% w/v of said alkyl urea polyalkylene imine polymer. The coating composition may favourably further comprise between about 0.02-6% w/v, or between about 0.05-4% w/v, or between about 0.04-5.0% w/v, or between about 0.06-4.0% w/v or between about 0.1-1% w/v, or between about 0.3-8% w/v, of additional cationic polymer(s). The liquid coating composition may, for example, comprise a blend of an alkyl urea polyalkylene imine and an anionic component such as an anionic polymer, wherein the composition comprises between about 0.02-5% w/v, especially between about 0.1-5% w/v, of said alkyl urea polyalkylene imine. Alternatively, the liquid coating composition may, for example, comprise a blend of an alkyl urea polyalkylene imine, an anionic component such as an anionic polymer and one or more additional cationic polymer(s), such as a polyalkylene imine, wherein the composition comprises between about 0.02-0.9% w/v of said alkyl urea polyalkylene imine.

In some embodiments where the coating composition comprises both an alkyl urea polyalkylene imine polymer, such as a butyl urea polyethylene imine polymer, and an additional polyalkylene imine polymer, especially an unsubstituted polyalkylene imine polymer, such as an unsubstituted polyethylene imine polymer, the liquid coating composition may suitably comprise at least about 0.005% w/v, or at least about 0.01% w/v, or at least about 0.02% w/v, or at least about 0.03% w/v of said alkyl urea polyalkylene imine polymer; and/or may suitably comprise no more than about 25% w/v, or no more than about 20% w/v, or no more than about 15% w/v, or no more than about 10% w/v, or no more than about 8% w/v, or no more than about 7% w/v, or no more than about 6% w/v, of said alkyl urea polyalkylene imine. The liquid coating composition may also suitably comprise at least about 0.03% w/v, or at least about 0.05% w/v, or at least about 0.1% w/v, or at least about 0.3% w/v, or at least about 0.5% w/v, of said additional polyalkylene imine polymer; and/or may suitably comprise no more than about 25% w/v, or no more than about 20% w/v, or no more than about 15% w/v, or no more than about 10% w/v, or no more than about 8% w/v, or no more than about 6% w/v, or no more than about 4% w/v, or no more than about 3.5% w/v, of said additional polyalkylene imine polymer such as said substituted or unsubstituted polyethylene imine polymer. In particular, the composition may comprise between about 0.01-7% w/v, favourably between about 0.03-3.5% w/v of alkyl urea polyalkylene imine and may comprise between about 0.01-10% w/v, or between about 0.04-5% w/v, favourably between about 0.06-4% w/v of additional polyalkylene imine polymer(s).

In some embodiments, a coating or liquid coating composition according to the present disclosure may comprise an alkyl urea polyalkylene imine and additional cationic polymer(s) in a w/w ratio in the range 1:50-5:1; suitably, in the range 1:50-1:1. Suitably, the w/w ratio of alkyl urea polyalkylene imine:additional cationic polymer(s) may be no less than 1:50, or 1:40, or 1:30, or 1:25, or 1:20, or 1:15, or 1:10; and/or may suitably be no more than 5:1, or 4:1, or 3:1, or 2:1, or 1:1, or 1:2, or 1:3, or 1:4, or 1:5. Suitably, this w/w ratio may be in the range 1:1-1:10.

A liquid coating composition according to the present disclosure may suitably comprise at least about 0.001% w/v, or at least about 0.002% w/v, or at least about 0.003% w/v, or at least about 0.004% w/v, or at least about 0.005% w/v of said anionic component. The composition may suitably comprise no more than about 0.5% w/v, or no more than about 0.3% w/v, or no more than about 0.25% w/v, or no more than about 0.2% w/v, or no more than about 0.18% w/v, or no more than about 0.15% w/v, or no more than about 0.12% w/v, or no more than about 0.10% w/v, or no more than about 0.09% w/v, or no more than about 0.08% w/v, or no more than about 0.07% w/v, or no more than about 0.06% w/v, or no more than about 0.05% w/v, or no more than about 0.02% w/v, of said anionic component. In particular, the coating composition may favourably comprise between about 0.001-0.20% w/v or between about 0.001-0.06% w/v of said anionic component; or between about 0.005-0.10% w/v or between about 0.005-0.03% w/v or between about 0.005-0.3% w/v of said anionic component.

In some embodiments of the liquid coating composition and/or the coating, the w/w ratio of the combined quantity of alkyl urea polyalkylene imine and any additional cationic polymer(s) in relation to the quantity of anionic component is in the range 500:1-15:1, suitably in the range 500:1-30:1. Suitably, the w/w ratio of the combined quantity of alkyl urea polyalkylene imine and any additional cationic polymer(s) to the quantity of anionic component may be no more than 500:1, or 400:1, or 300:1, or 250:1, or 200:1, or 100:1, or 95:1, or 90:1, or 85:1, or 80:1, or 75:1; and/or may suitably be no less than 15:1, or 20:1, or 25:1, or 30:1, or 40:1, or 50:1, or 60:1, or 65:1. Suitably, this w/w ratio may be in the range 30:1-70:1.

In embodiments which include a guanidine compound, a liquid coating composition according to the present disclosure may suitably comprise at least about 0.2% w/v, or at least about 0.3% w/v, or at least about 0.4% w/v, or at least about 0.5% w/v, or at least about 0.8% w/v, or at least about 1% w/v, or at least about 1.5% w/v of said guanidine compound. The composition may suitably comprise no more than about 10% w/v, or no more than about 8% w/v, or no more than about 7% w/v, or no more than about 6% w/v, or no more than about 5% w/v, or no more than about 4% w/v, or no more than about 3.5% w/v, or no more than about 3% w/v, of said guanidine compound. In particular, the liquid coating composition or coating may comprise between about 0.5-30.5% w/v, or between about 0.5-3% w/v of said guanidine compound; or between about 1-5.5% w/v of said guanidine compound.

In a liquid coating composition according to the present disclosure which does not contain a guanidine compound, the total quantity of alkyl urea polyalkylene imine, anionic component and (where present) additional cationic polymer(s) in the composition may optionally be no more than about 25% w/v, or no more than about 20% w/v, or no more than about 15% w/v, or no more than about 10% w/v of the composition; suitably no more than about 9% w/v of the composition, or about 8% w/v of the composition, or about 7% w/v of the composition, or about 6% w/v of the composition, or about 5% w/v of the composition.

In a liquid coating composition according to the present disclosure which does not contain a guanidine compound, the total quantity of alkyl urea polyalkylene imine, anionic component and (where present) additional cationic polymer(s) in the composition may suitably be at least about 0.05% w/v of the composition; suitably at least about 0.1% w/v of the composition, or about 0.2% w/v of the composition; for example at least about 0.25% w/v of the composition, or about 0.5% w/v of the composition; or suitably at least about 1% w/v of the composition, or about 2% w/v of the composition, or about 3% w/v of the composition.

In a liquid coating composition according to the present disclosure which does contain a guanidine compound, the total quantity of alkyl urea polyalkylene imine, guanidine compound, anionic component and (where present) additional cationic polymer(s) may optionally be no more than about 25% w/v of the composition, or no more than about 20% w/v of the composition, or no more than about 15% w/v of the composition; suitably no more than about 12% w/v of the composition, or about 10% w/v of the composition, or about 9% w/v of the composition, or about 8% w/v of the composition. The total quantity of alkyl urea polyalkylene imine, guanidine compound, anionic component and (where present) additional cationic polymer(s) may optionally be at least about 0.5% w/v of the composition; suitably at least about 1% w/v of the composition, or about 1.5% w/v of the composition, or about 2% w/v of the composition.

A liquid coating composition according to the present disclosure may optionally comprise:
0.02-4.5% w/v of alkyl urea polyalkylene imine;
0.001-0.20% w/v of anionic component, such as anionic polymer, such as polyacrylic acid; and
optionally 0.1-5% w/v of additional cationic polymer(s), e.g. polyethylene imine.

For example, the liquid coating composition according to the present disclosure may optionally comprise:
0.01-0.9% w/v of alkyl urea polyalkylene imine;
0.001-0.20% w/v of anionic component such as anionic polymer; and
0.1-5% w/v of additional cationic polymer(s).

The liquid coating composition according to the present disclosure may optionally comprise:
0.03-4.5% w/v of alkyl urea polyalkylene imine; and
0.001-0.20% w/v of anionic component such as anionic polymer.

A liquid coating composition according to the present disclosure may optionally comprise:
0.05-3.5% w/v of guanidine compound;
0.01-7.0% w/v of alkyl urea polyalkylene imine;
0.002-0.1% w/v of anionic component such as anionic polymer; and
optionally 0.05-4% w/v of additional cationic polymer(s).

Alternatively, a liquid coating composition according to the present disclosure may optionally comprise:
0.1-3.0% w/v of guanidine compound;
0.3-4.5% w/v of alkyl urea polyalkylene imine; and
0.009-0.05% w/v of anionic component such as anionic polymer.

A liquid coating composition according to the present disclosure, which may be particularly suitable for application to living substrates such as skin, and may be suitable for use as a skin sanitiser product, may suitably comprise:
0.01-2% w/v of alkyl urea polyalkylene imine;
0.005-0.3% w/v of anionic component such as anionic polymer such as polyacrylic acid;
0.1-1% w/v of additional cationic polymer such as polyethylene imine; and optionally
0.001-0.2% w/v of benzalkonium chloride and/or benzethonium chloride;
where these components total 0.1-4% w/v of the composition.

A liquid coating composition according to the present disclosure, which may be particularly suitable for application to porous or non-porous surfaces, and may be suitable for use on inert (non-living) surfaces, may suitably comprise:
0.05-7% w/v of alkyl urea polyalkylene imine;
0.3-8% w/v of additional cationic polymer such as polyethylene imine;
0.5-3.5% w/w of guanidine compound; and optionally
0.001-0.2% w/v of benzalkonium chloride and/or benzethonium chloride, and/or 0.005-0.3% w/v of anionic component such as anionic polymer such as polyacrylic acid;
where these components total 1-19% w/v of the composition.

These components may be blended in a liquid medium such as an aqueous/alcoholic medium, optionally together with one or more additional ingredients, such as a cross-linking agent as herein defined. For example, one or more additional anti-microbial agents, such as an additional anti-microbial cationic component, such as a quaternary ammonium salt, such as benzalkonium chloride and/or benzethonium chloride, may optionally be included in the blended composition, optionally in an amount between about 0.001-1% w/v, optionally between about 0.005-1.0% w/v, for example between about 0.01-0.05% w/v.

Optionally, the liquid coating composition may further comprise one or more binders, which are effective to improve adhesion of the coating composition to the coated surface. The binders may, for example, be polyamine, polyacrylate and/or polyurethane binders, as disclosed, for example, in US 2021/0156080. The binders may, in particular, comprise polyurethane, and/or an acrylic co-polymer emulsion, and/or a polyurethane dispersion, and/or a multifunctional acrylate. The binder may be mixed or blended into the liquid coating composition. The binder may be included in the composition in a quantity of about 0.1-30% w/v, optionally 1-20% w/v, or 2-10% w/v, optionally 3-8% w/v or 0.1-2.5% w/v. Inclusion of a binder in the liquid coating composition may be particularly advantageous where the composition is used or is to be used for coating fabrics or textiles, or for coating other porous surfaces; as discussed in Wang et al, Coatings 2020(10) 520.

The liquid coating composition may further comprise one or more surfactants, such as but not limited to anionic surfactants including sulphates, sulfonates or gluconates, including sodium dodecyl sulphate; non-ionic surfactants including cocamide, ethoxylates or alkoxylates; cationic surfactants including alkyl ammonium chloride; amphoteric surfactants including betaines and amino oxides; and surfactants which also have anti-microbial activity, such as cationic surfactants, such as benzalkonium chloride and benzethonium chloride; and cetrimonium bromide.

In some embodiments, the liquid coating composition may be a liquid disinfectant, which is suitable for application to substrates formed from inert (non-living) materials, including porous and non-porous surfaces. In other embodiments, the liquid coating composition may be a liquid antiseptic or sanitiser, which is suitable for application to body parts of living humans or animals, such as to the skin or hair of living humans or animals. A liquid antiseptic or sanitiser in accordance with the present disclosure may further comprise additional ingredients suitable for application to the body, in particular to the skin or to the hair; such as glycerol and/or ceramides and/or hyaluronic acid and/or moisturising agents and/or fragrances. A liquid antiseptic or skin sanitiser in accordance with the disclosure may further comprise one or more natural emollients; such as triglycerides or short/medium chain fatty acids, including but not limited to stearic acid, linoleic acid, oleic acid and lauric acid; hydrocarbons, including but not limited to mineral oil, petrolatum and paraffin; and natural esters, including but not limited to lanolin. A liquid antiseptic or skin sanitiser in accordance with the disclosure may further comprise one or more synthetic emollients, including but not limited to esters and alcohols. A liquid antiseptic or skin sanitiser in accordance with the disclosure may further comprise one or more humectants, including but not limited to glycerine, hyaluronic acid, gelatin, urea and sorbitol. A liquid antiseptic or sanitiser or disinfectant in accordance with the present disclosure may suitably comprise isopropyl alcohol, ethanol or propanol, and/or water; and may in particular comprise an alcohol and water. A liquid antiseptic or sanitiser or disinfectant in accordance with the present disclosure may optionally comprise one or more additional anti-microbial agents, such as one or more additional anti-microbial cationic components, such as a quaternary ammonium salt, such as benzalkonium chloride and/or benzethonium chloride, optionally in a quantity of between about 0.001-1% w/v, optionally between about 0.005-1.0% w/v such as about 0.01% w/v to about 0.05% w/v.

In some preferred embodiments, the present disclosure provides a liquid coating composition in accordance with any of the following numbered statements 1-17:

1. A liquid coating composition suitable for forming an anti-microbial coating comprising an alkyl urea polyalkylene imine, an anionic polymer and optionally a cationic polymer; the liquid coating composition comprising a blend of an alkyl urea polyalkylene imine, an anionic polymer, and optionally a cationic polymer.

2. A liquid coating composition according to statement 1, wherein the blend is formulated as a solution, suspension, dispersion, or emulsion, in a liquid medium which is aqueous, alcoholic, aqueous/alcoholic, or organic solvent.

3. A liquid coating composition according to statement 2, wherein the liquid medium comprises methanol, ethanol, propanol and/or isopropanol.

4. A liquid coating composition according to any of statements 1-3, comprising at least about 0.02% w/v, or at least about 0.03% w/v, or at least about 0.05% w/v, or at least about 0.08% w/v, or at least about 0.10% w/v of said alkyl urea polyalkylene imine.

5. A liquid coating composition according to any of statements 1-4, comprising no more than about 10% w/v, or no more than about 8% w/v, or no more than about 7% w/v, or no more than about 6% w/v, or no more than about 5% w/v, of said alkyl urea polyalkylene imine, or no more than about 0.9% w/v, or comprising no more than about 0.75% w/v, or no more than about 0.7% w/v, or no more than about 0.6% w/v, or no more than about 0.5% w/v, of said alkyl urea polyalkylene imine.

6. A liquid coating composition according to any of statements 1-5, comprising between about 0.01-0.9% w/v, or between about 0.1-5% w/v, of said alkyl urea polyalkylene imine.

7. A liquid coating composition according to any of statements 1-6, comprising at least about 0.001% w/v, or at least about 0.002% w/v, or at least about 0.003% w/v, or at least about 0.004% w/v, or at least about 0.005% w/v of said anionic polymer.

8. A liquid coating composition according to any of statements 1-7, comprising no more than about 0.3% w/v, or no more than about 0.25% w/v, or no more than about 0.2% w/v, or no more than about 0.18% w/v, or no more than about 0.15% w/v, or no more than about 0.12% w/v, or no more than about 0.10% w/v, of said anionic polymer.

9. A liquid coating composition according to any of statements 1-8, comprising between about 0.001-0.20% w/v of said anionic polymer; suitably comprising between about 0.005-0.10% w/v of said anionic polymer.

10. A liquid coating composition according to any of statements 1-9, comprising at least about 0.01% w/v, or at least about 0.05% w/v, or at least about 0.1% w/v, or at least about 0.15% w/v, or at least about 0.2% w/v of said cationic polymer.

11. A liquid coating composition as claimed in any of claims according to any of statements 1-10, comprising no more than about 10% w/v, or no more than about 8% w/v or no more than about 7% w/v, or no more than about 6% w/v, or no more than about 5% w/v, or no more than about 4% w/v, of said optional cationic polymer.

12. A liquid coating composition according to any of statements 1-11, comprising between about 0.1-10% w/v of said optional cationic polymer; suitably comprising between about 0.15-7% w/v or between about 0.2-5% w/v of said cationic polymer.

13. A liquid coating composition according to any of statements 1-12, wherein the w/w ratio of cationic polymer:alkyl urea polyalkylene imine is in the range 50:1-1:1; suitably no more than 50:1, or 40:1, or 30:1, or 25:1, or 20:1, or 15:1, or 10:1; suitably no less than 1:1, or 2:1, or 3:1, or 4:1, or 5:1.

14. A liquid coating composition according to any of statements 1-13, wherein the w/w ratio of the combined quantity of optional cationic polymer and alkyl urea polyalkylene imine to the quantity of anionic polymer is in the range 100:1-30:1; suitably no more than 100:1, or 95:1, or 90:1, or 85:1, or 80:1, or 75:1; suitably no less than 30:1, or 40:1, or 50:1, or 60:1, or 65:1.

15. A liquid coating composition according to any of statements 1-14, wherein the total quantity of said optional cationic polymer, said alkyl urea polyalkylene imine, and said anionic polymer in the composition is no more than about 10% w/v of the composition; suitably no more than about 9% w/v of the composition, or about 8% w/v of the composition, or about 7% w/v of the composition, or about 6% w/v of the composition, or about 5% w/v of the composition.

16. A liquid coating composition according to any of statements 1-15, wherein the total quantity of said optional cationic polymer, said alkyl urea polyalkylene imine, and said anionic polymer in the composition is at least about 0.05% w/v of the composition; suitably at least about 0.1% w/v of the composition, or about 0.2% w/v of the composition.

17. A liquid coating composition according to any of statements 1-16, wherein the composition comprises:
0.03-4.5% w/v of said alkyl urea polyalkylene imine;
0.001-0.1% w/v of said anionic polymer; and
optionally 0.1-5% w/v of said cationic polymer.

In other preferred embodiments, the present disclosure provides a liquid coating composition in accordance with any of the following numbered statements 1-25:

1. A liquid coating composition suitable for forming an anti-microbial coating comprising a guanidine compound, an alkyl urea polyalkylene imine polymer, an anionic polymer and optionally a further cationic polymer; the liquid coating composition comprising a blend of a guanidine compound, an alkyl urea polyalkylene imine polymer, an anionic polymer and optionally a further cationic polymer.

2. A liquid coating composition according to statement 1, wherein the blend is formulated as a solution, suspension, dispersion, or emulsion, in a liquid medium which is aqueous, alcoholic, aqueous/alcoholic, or organic.

3. A liquid coating composition according to statement 2, wherein the liquid medium comprises methanol, ethanol, propanol and/or isopropanol.

4. A liquid coating composition according to any of statements 1-3, wherein the alkyl urea polyalkylene imine polymer is a polyethylene imine or a polypropylene imine.

5. A liquid coating composition according to any of statements 1-4, wherein the alkyl urea polyalkylene imine polymer is either:
substituted with one or more straight chain, branched or cyclic alkyl groups, selected from methyl, ethyl, propyl, butyl, pentyl, hexyl, heptyl, octyl, nonyl or decyl groups, via a urea linkage, favourably one or more ethyl, propyl, butyl, or pentyl groups; or
which is substituted with one or more alkyl groups comprising an alkylene chain linked to the polyalkylene imine with two or more urea linkages; especially 1-10 methylene groups via a urea linkage at either end of an alkylene chain, favourably 3-7 methylene groups.

6. A liquid coating composition according to any of statements 1-5, wherein the guanidine compound comprises a cross-linkable polymer bearing one or more guanidine or biguanidine groups, and wherein the liquid coating composition further comprises a cross-linking agent for cross-linking the polymer.

7. A liquid coating composition according to statement 6, wherein the guanidine compound comprises a cross-linkable polymer bearing one or more cross-linkable carboxylic acid groups.

8. A liquid coating composition according to statement 6 or statement 7, wherein the cross-linking agent is an aziridine compound such as a polyaziridine compound.

9. A liquid coating composition according to any of statements 1-8, comprising at least about 0.01% w/v, or at least about 0.02% w/v, or at least about 0.05% w/v, or at least about 0.1% w/v, or at least about 0.2% w/v of said alkyl urea polyalkylene imine polymer.

10. A liquid coating composition according to any of statements 1-9, comprising no more than about 10% w/v, or no more than about 8% w/v, or no more than about 7% w/v, or no more than about 6.5% w/v, or no more than about 6% w/v, of said alkyl urea polyalkylene imine polymer.

11. A liquid coating composition according to any of statements 1-10, comprising between about 0.01-7% w/v, or between about 0.1-6% w/v, of said alkyl urea polyalkylene imine polymer.

12. A liquid coating composition according to any of statements 1-11, comprising at least about 0.001% w/v, or at least about 0.002% w/v, or at least about 0.003% w/v, or at least about 0.004% w/v, or at least about 0.005% w/v of said anionic polymer.

13. A liquid coating composition according to any of statements 1-12, comprising no more than about 0.03% w/v, or no more than about 0.025% w/v, or no more than about 0.02% w/v, or no more than about 0.018% w/v, or no more than about 0.015% w/v, or no more than about 0.012% w/v, or no more than about 0.01% w/v, of said anionic polymer.

14. A liquid coating composition according to any of statements 1-13, comprising between about 0.001-0.03% w/v of said anionic polymer; suitably comprising between about 0.005-0.015% w/v of said anionic polymer.

15. A liquid coating composition according to any of statements 1-14, comprising at least about 0.5% w/v, or at least about 0.8% w/v, or at least about 1% w/v, or at least about 1.5% w/v, or at least about 2% w/v of said guanidine compound.

16. A liquid coating composition according to any of statements 1-15, comprising no more than about 10% w/v, or no more than about 8% w/v, or no more than about 7% w/v, or no more than about 6% w/v, or no more than about 5% w/v, or no more than about 4% w/v, or no more than about 3% w/v, of said guanidine compound.

17. A liquid coating composition according to any of statements 1-16, comprising between about 0.5-10% w/v of said guanidine compound; suitably comprising between about 1-3% w/v of said guanidine compound.

18. A liquid coating composition according to any of statements 1-17, wherein the optional cationic polymer is a further polyalkylene imine polymer, such as an unsubstituted polyalkylene imine polymer.

19. A liquid coating composition according to any of statements 1-18, comprising at least about 0.1% w/v, or at least about 0.02% w/v, or at least about 0.04% w/v, or at least about 0.05% w/v, or at least about 0.1% w/v of said optional further cationic polymer.

20. A liquid coating composition according to any of statements 1-19, comprising no more than about 6% w/v, or no more than about 5% w/v or no more than about 4% w/v, or no more than about 3.5% w/v, or no more than about 3% w/v of said optional further cationic polymer.

21. A liquid coating composition according to any of statements 1-20, comprising between about 0.02-6% w/v of said further polyalkylene imine; suitably comprising between about 0.05-4% w/v of said optional further cationic polymer.

22. A liquid coating composition according to any of statements 1-21, wherein the total quantity of said guanidine compound, said optional further cationic polymer, said alkyl urea polyalkylene imine polymer, and said anionic polymer in the composition is no more than about 15% w/v of the composition; suitably no more than about 12% w/v of the composition, or about 10% w/v of the composition, or about 9% w/v of the composition, or about 8% w/v of the composition.

23. A liquid coating composition according to any of statements 1-22, wherein the total quantity of said guanidine compound, said optional further cationic polymer, said alkyl urea polyalkylene imine polymer, and said anionic polymer in the composition is at least about 0.7% w/v of the composition; suitably at least about 1% w/v of the composition, or about 1.5% w/v of the composition, or about 2% w/v of the composition.

24. A liquid coating composition according to any of statements 1-23, wherein the composition comprises:
0.05-3.5% w/v of said guanidine compound;
0.01-7.0% w/v of said alkyl urea polyalkylene imine polymer;
0.002-0.1% w/v of said anionic polymer; and
0.05-4% w/v of optional further cationic polymer.

25. A liquid coating composition according to any of statements 1-23, wherein the composition comprises:
0.1-3.0% w/v of said guanidine compound;
0.3-4.5% w/v of said alkyl urea polyalkylene imine polymer; and
0.009-0.05% w/v of said anionic polymer.

The present disclosure embraces methods of providing an anti-microbial coating in accordance with this disclosure on a substrate or article. These may also be understood to be methods of coating a substrate or article with an anti-microbial coating in accordance with this disclosure.

In some embodiments, the substrate or article may be formed from one or more inert (non-living) materials. The inert material or materials may include a natural material, such as wood or stone; or may include an artificial material, such as a plastic material. The inert material or materials may include a porous material, such as a porous textile or fabric. The inert material or materials may include a non-porous material, such as metal or glass. The substrate or article may comprise a combination of inert materials, which may be porous and/or non-porous; such as a metal substrate clad with a porous fabric. It will be understood that when a porous article or substrate, such as a fabric or textile, is coated according to the present disclosure, the coating or coating composition may to some extent seep or penetrate through and/or within the porous structure of the article or substrate; that is, may not remain solely on the surface of the article or substrate. A porous article or substrate that is coated according to the present disclosure may therefore be, in effect, impregnated and/or wholly or partly saturated with the coating according to the disclosure.

The inert material or materials may, in particular, comprise one or more of a plastic material, an elastomeric material, such as an elastane, such as Spandex® or Lycra®, or a synthetic rubber, and/or a polymeric material. The inert material or materials may, for example, comprise a polyurethane such as carbothane polyurethane; a silica or silicone material such as polydimethylsiloxane and/or a polyester-polysiloxane; a polyester such as polyethylene such as polyethylene terephthalate and/or polytetrafluoroethylene, and/or polypropylene; a polycarbonate; a polyamide, polyamine and/or polyimine and/or polyimide including nylon; latex; nitrile; polyisoprene; a polyvinyl polymer including a polyacrylate, a polymethacrylate such as polymethyl methacrylate and/or a hydroxyethyl methacrylate polymer, a polyacrylamide, a polymethacrylamide, polyvinyl chloride and/or polyvinylidene fluoride; and/or a polyimide polymer.

The inert material or materials may comprise natural and/or synthetic biopolymers. Natural biopolymers include collagen, silk fibroin, starch, cellulose and chitosan. Synthetic biopolymers include polylactic acid, polyglycolic acid, and polyethylene glycol. The inert material or materials may comprise bioabsorbable materials such as polyglycolic acid and/or poly-L-lactic acid, polycaprolactone, poly-DL-lactic acid, poly(trimethylene carbonate), and/or poly(para-dioxanone).

The inert material or materials may comprise a metal and/or metal alloy such as titanium, nickel, titanium-nickel alloy, cobalt-chrome alloy, gold, platinum, silver, iridium, tantalum, tungsten and/or steel including stainless steel. The inert material or materials may comprise a ceramics material; and/or glass; and/or an organic material including an animal-derived material such as collagen and/or a decellularised graft; or mixtures and combinations thereof, including, for example, organic substrates with a metal scaffold.

The inert material or materials may comprise a fabric or textile, such as a woven or non-woven fabric, including natural or synthetic fabric or textile material; including a melt-blown polymer material, or nylon or rayon or polyester or polyester cellulose or polyethylene or polypropylene, or leather, or silk, or cotton.

An article formed from one or more inert materials and coated in accordance with the present disclosure may be an object which is used or is typically present in a clinical setting, or which is used or useful for any clinical purpose, including for diagnostic or therapeutic purposes. Thus, for example, an article coated in accordance with the present disclosure may be a medical device such as a catheter or implant; or a medical implement such as a dish, tray, operating table or spatula; or an item of personal protective equipment (PPE) such as a mask, gown, apron or gloves, or a face shield, or medical scrubs, or surgical gowns, or eye protection; or an item for patient use, such as a table, chair, bed or commode; or an item for use in cleaning, purification or sterilisation, such as a cloth, sponge, filter or wipe. Substrates and articles coated according to this disclosure include medical devices and implants used in the healthcare (including dentistry) and veterinary sectors; including catheters, endoscopes, cardiac implants such as stents, heart valves and biodegradable, non-biodegradable, natural and/or synthetic scaffolds and/or grafts, bone and joint implants, surgical tools and other types of diagnostic, surgical and therapeutic equipment. An article coated in accordance with the present disclosure may, in particular, be a surgical mask (Type IIR, FFP1) or a surgical gown. An article coated in accordance with the present disclosure may be any surface in a clinical environment, such as a wall, door handle, or screen; or an item of equipment used in a clinical environment such as a baby incubator or an item of therapeutic or diagnostic equipment; or may be a plastic film which is shaped and configured for application and attachment to a surface in a clinical environment as herein defined.

A substrate formed from one or more inert materials and coated in accordance with the present disclosure may be an object or surface which is regularly touched or handled by people, such as (but without limitation) a wall, door or window, or a handle of a door or window; or the textile or other surface of a seat, or a curtain, cloth, item of clothing, or bed sheet; or a support or guard rail or hand rail, or a counter, table, desk, floor, chair or bed; or a surface of any object, including furniture and/or fittings, in a public or semi-public place including public transport including trains or aeroplanes, or an institution including an educational institution such as a school, college, or university, or a healthcare institution such as a hospital, medical centre, dentistry, vet or a surgery, or a government or local council centre, courthouse or prison, or a private or semi-private venue including a shop, entertainment centre, restaurant, or private home; or a commercial or military setting such as transportation including ships or submarines; or a consumer item such as a phone, phone cover, phone screen, toy, or item of crockery or cutlery; or a cover or a plastic film, such as a label, which is shaped and/or configured for application and/or attachment to an object or surface which is regularly touched or handled by people, as herein defined.

In some embodiments, the substrate may be a part of the human or animal body, particularly the skin of a human or animal, such as the hands or face or feet of a human. The present disclosure accordingly provides methods for applying an anti-microbial coating in accordance with this disclosure to the body, including the skin, of a human or animal.

In one aspect of the disclosure, a method of providing an anti-microbial coating to a substrate or article in accordance with this disclosure may comprise applying a liquid coating composition in accordance with this disclosure to the surface of the substrate or article. The liquid coating composition may be applied to the surface either once or in multiple applications, such as two applications or more than two applications.

In this aspect, the method of providing an anti-microbial coating in accordance with this disclosure on a substrate or article may, for example, include incubating the substrate or article in the liquid coating composition, and/or immersing the substrate or article in the liquid coating composition, and/or washing the substrate or article with the liquid coating composition, and/or dipping the substrate or article into the liquid coating composition either once or more than once, and/or causing the liquid coating composition to flow over the substrate or article, and/or spraying the liquid coating composition onto the substrate or article, and/or painting the liquid coating composition onto the substrate or article, and/or wiping the liquid coating composition onto the substrate or article, and/or brushing and/or padding and/or rolling the liquid coating composition onto the substrate or article, and/or applying the liquid coating composition to the substrate or article using any other application technique, including physical deposition and/or electrophoretic deposition, where these application techniques may optionally be used in any sequence or combination and may optionally be performed or repeated once or more than once. This method may be used for providing an anti-microbial coating on any substrate or article, including substrates which are part of the human or animal body, such as human or animal skin; and substrates and articles which are not part of the human or animal body, including substrates and articles formed from natural and/or artificial materials, and/or from porous and/or non-porous materials.

The method of this aspect of the disclosure may further include a step of drying and/or curing the coating on the substrate or article. In particular, the coating may be dried and/or cured or allowed to dry and/or cure on the substrate or article between applications of the liquid coating composition, or following the or all applications of the liquid coating composition to the substrate or article.

Optionally, therefore, the method of this aspect of the disclosure may include heat-curing the coating on the substrate or article, optionally by placing the substrate or article in an environment heated to a temperature of at least about 35° C., or to at least about 50° C., for example to a temperature of about 140° C., preferably to a temperature of about 60-100° C. This may be particularly suitable where the substrate or article is not a part of a human or animal body and/or is not formed from a natural or heat-sensitive material. Alternatively or additionally, the method may further comprise a step of UV-curing the coating on the substrate or article, using methods known in the art. For example. the UV version of guanidine GC (example 1b) can be cured using UV.

The method of this aspect of the disclosure may further include a step of applying a binder composition to the substrate or article. The binder composition may, for example, comprise a polyamine, polyacrylate and/or polyurethane binder, as disclosed, for example, in US 2021/0156080; where the binder or binders may be effective to improve adhesion of the coating to the coated surface. The binder composition may be applied to the substrate or article in liquid form, for example as a solution, suspension, emulsion or dispersion. The binder composition may, in particular, comprise polyurethane, and/or an acrylic co-polymer emulsion, and/or a polyurethane dispersion, and/or a multifunctional acrylate. The method of this aspect of the disclosure may accordingly include a step of applying a binder composition as disclosed to the substrate or article before or after applying the liquid coating composition to the substrate or article. In some embodiments, the method may include a step of applying the liquid coating composition to the substrate or article, followed by a step of applying the binder composition to the substrate or article, followed by a step of applying the liquid coating composition to the substrate or article. The step of applying the binder composition to the substrate or article may comprise incubating the substrate or article in the binder composition, and/or immersing the substrate or article in the binder composition, and/or washing the substrate or article with the binder composition, and/or dipping the substrate or article into the binder composition either once or more than once, and/or causing the binder composition to flow over the substrate or article, and/or spraying the binder composition onto the substrate or article, and/or painting the binder composition onto the substrate or article, and/or wiping the binder composition onto the substrate or article, and/or brushing and/or padding and/or rolling the binder composition onto the substrate or article, and/or applying the binder composition to the substrate or article using any other suitable application technique or any combination of application techniques, including physical deposition and/or electrophoretic deposition. Optionally, the method includes a step of drying or allowing the coated article or substrate to dry, after the binder composition is applied.

This aspect of the disclosure accordingly provides a method of coating an article which is a medical device, such as an implantable medical device, where the step of applying the liquid coating composition comprises dipping the medical device into the liquid coating composition, washing and/or rinsing the medical device with the liquid coating composition; and/or spraying, painting, rubbing, padding, rolling and/or brushing the liquid coating composition onto the medical device; and/or applying the liquid coating composition to the medical device by physical deposition and/or electrophoretic deposition. Optionally, the method may further comprise drying the coated medical device or allowing the coated medical device to dry, optionally at room temperature or in a thermal oven, and or curing the coating on the medical device, for example, using heat or by exposing to UV radiation. Optionally, the method may further comprise applying a binder composition to the medical device, before and/or after applying the liquid coating composition to the medical device.

This aspect of the disclosure further provides a method of coating a porous substrate, such as a textile or fabric substrate, where the step of applying the liquid coating composition comprises dipping the porous substrate into the liquid coating composition, washing and/or rinsing the porous substrate with the liquid coating composition; and/or spraying, painting, rubbing, padding, rolling and/or brushing the liquid coating composition onto the porous substrate; and/or depositing the liquid coating composition onto the porous substrate; where these application steps may optionally be performed in any combination or sequence and may optionally be performed or repeated once or more than once. Optionally, the method may further comprise drying the coated porous substrate or allowing the coated porous substrate to dry, optionally at room temperature, or in a thermal oven, and or curing the coating on the medical device, for example, using heat or by exposing to UV radiation. Optionally, the method may further comprise applying a binder composition to the porous substrate, before and/or after applying the liquid coating composition to the porous substrate.

This aspect of the disclosure also provides a method of coating a substrate which is a body part of a living human or animal, such as the skin of a living human or animal, where the step of applying the liquid coating composition to the substrate comprises washing and/or rinsing the body part with the liquid coating composition; and/or spraying, rubbing, padding, rolling, depositing and/or brushing the liquid coating composition onto the body part. Optionally, the method may further comprise drying the coated body part or allowing the coated body part to dry, optionally at room temperature.

In another aspect, the disclosure provides a method of providing an anti-microbial coating in accordance with this disclosure on a substrate or article using a “layering” approach, which involves sequential application of separate liquid compositions each comprising one or more of the components of the anti-microbial coating described herein; optionally, in the quantities disclosed herein. This may be particularly suitable for application of the coating to substrates or articles which are not part of the human or animal body. It may be particularly suitable for application of the coating to substrates or articles which are formed from non-living or artificial materials.

The present disclosure accordingly provides a method of providing an anti-microbial coating in accordance with the disclosure on a substrate or article, comprising the sequential steps of: (a) applying a first liquid composition comprising one or more of an alkyl urea polyalkylene imine polymer as herein defined, an anionic component such as an anionic polymer as herein defined, an additional cationic polymer(s) as herein defined and a guanidine compound as herein defined to the substrate or article one or more times to form a first layer; followed by (b) applying a second liquid composition different from the first liquid composition and comprising one or more of an alkyl urea polyalkylene imine polymer as herein defined, an anionic component such as an anionic polymer as herein defined, an additional cationic polymer(s) as herein defined and a guanidine compound as herein defined to the substrate or article one or more times to form a second layer; followed by (c) optionally repeating step (a) and/or step (b); such as to produce a coating comprising an alkyl urea polyalkylene imine polymer in accordance with the disclosure.

The method may further comprise. (d) applying a third and, optionally, subsequent liquid composition different from the first and/or second liquid compositions and comprising one or more of an alkyl urea polyalkylene imine polymer as herein defined, an anionic component such as an anionic polymer as herein defined, an additional cationic polymer(s) as herein defined and/or a guanidine compound as herein defined to the substrate or article one or more times to form a third layer and optionally one or more subsequent layers.

To yield a coating in accordance with this disclosure, at least one of the first, second and (where applicable) third or subsequent liquid compositions must typically comprise said alkyl urea polyalkylene imine polymer, optionally in an amount at least about 0.005% w/v, or at least about 0.01% w/v, or at least about 0.02% w/v, or at least about 0.03% w/v, or at least about 0.05% w/v, or at least about 0.08% w/v, or at least about 0.10% w/v, or at least about 0.15% w/v, or at least about 0.2% w/v; and/or no more than about 25% w/v, or no more than about 20% w/v, or no more than about 15% w/v, or no more than about 10% w/v, or no more than about 8% w/v, or no more than about 7% w/v, or no more than about 6.5% w/v, or no more than about 6% w/v, or no more than about 5% w/v; suitably in an amount between about 0.01-20% w/v; suitably between about 0.01-10% w/v or between about 0.05-10% w/v or between about 0.5-7% w/v. In some embodiments, at least one of said first, second and (where applicable) third or subsequent liquid compositions may comprise an anionic component such as an anionic polymer as herein disclosed, optionally in an amount at least about 0.001% w/v, or at least about 0.002% w/v, or at least about 0.003% w/v, or at least about 0.004% w/v, or at least about 0.005% w/v; and/or no more than about 0.5% w/v, or no more than about 0.3% w/v, or no more than about 0.25% w/v, or no more than about 0.2% w/v, or no more than about 0.18% w/v, or no more than about 0.15% w/v, or no more than about 0.12% w/v, or no more than about 0.10% w/v, or no more than about 0.09% w/v, or no more than about 0.08% w/v, or no more than about 0.07% w/v, or no more than about 0.06% w/v, or no more than about 0.05% w/v, or no more than about 0.02% w/v; optionally in an amount between about 0.001-0.5% w/v, or between about 0.001-0.3% w/v, or between about 0.005-0.1% w/v; such as to yield an anti-microbial coating comprising an alkyl urea polyalkylene imine and an anionic component such as an anionic polymer as herein disclosed. In some embodiments, at least one of said first, second and (where applicable) third or subsequent liquid compositions may comprise an additional cationic polymer(s) as herein disclosed, optionally in an amount at least about 0.01% w/v, or at least about 0.02% w/v, or at least about 0.04% w/v, or at least about 0.05% w/v, or at least about 0.1% w/v, or at least about 0.15% w/v, or at least about 0.2% w/v, or at least about 0.3% w/v, or at least about 0.5% w/v; and/or no more than about 25% w/v, or no more than about 20% w/v, or no more than about 15% w/v, or no more than about 10% w/v, or no more than about 8% w/v or no more than about 7% w/v, or no more than about 6% w/v, or no more than about 5% w/v, or no more than about 4% w/v; optionally in an amount between about 0.01-20% w/v or between about 0.1-10% w/v or between about 0.3-8% w/v or between about 0.15-7% w/v or between about 0.2-5% w/v; such as to yield an anti-microbial coating comprising an alkyl urea polyalkylene imine and an additional cationic polymer(s) as herein disclosed. In some embodiments, at least one of said first, second and (where applicable) third or subsequent liquid compositions may comprise a guanidine compound as herein disclosed, optionally in an amount at least about 0.2% w/v, or at least about 0.3% w/v, or at least about 0.4% w/v, or at least about 0.5% w/v, or at least about 0.8% w/v, or at least about 1% w/v, or at least about 1.5% w/v; and/or no more than about 10% w/v, or no more than about 8% w/v, or no more than about 7% w/v, or no more than about 6% w/v, or no more than about 5% w/v, or no more than about 4% w/v, or no more than about 3.5% w/v, or no more than about 3% w/v; suitably in an amount between about 0.1-10% w/v, or between about 0.1-5% w/v, or between about 0.5-30.5% w/v, or between about 0.5-3% w/v of said guanidine compound; or between about 1-5.5% w/v; such as to yield an anti-microbial coating comprising an alkyl urea polyalkylene imine and a guanidine compound as herein disclosed.

In some embodiments, the first coating solution comprises an alkyl urea polyalkylene imine polymer; and the second coating solution comprises an alkyl urea polyalkylene imine polymer, an additional cationic polymer such as a polyalkylene imine polymer, and a guanidine compound. Optionally, the second coating solution may further comprise an anionic component as herein disclosed. Optionally, the first coating solution and/or the second coating solution may comprise one or more binders as herein disclosed.

In one favoured embodiment, the first coating solution comprises 0.05-7% w/v of alkyl urea polyalkylene imine; and the second coating solution comprises 0.05-7% w/v of alkyl urea polyalkylene imine, 0.3-8% w/v of additional cationic polymer such as polyethylene imine, and 0.5-3.5% w/w of guanidine compound.

In some preferred embodiments of this aspect of the disclosure, step (a) may comprise applying a first liquid composition comprising an alkyl urea polyalkylene imine polymer to the substrate or article one or more times to form a first layer. Step (b) may comprise applying a second liquid composition comprising an alkyl urea polyalkylene imine polymer and an additional cationic polymer such as an unsubstituted polyalkylene imine polymer to the substrate or article one or more times to form a second layer. Optionally, the second liquid composition may further comprise a guanidine compound and/or an anionic component such as an anionic polymer.

Each of the first liquid composition, the second liquid composition and/or the third and/or subsequent liquid composition may be formulated in a liquid medium as a solution, suspension, dispersion, or emulsion. The liquid medium may be aqueous, alcoholic, or aqueous/alcoholic. The liquid medium may comprise an organic solvent, such as a polar organic solvent. In some embodiments, the liquid medium may comprise methanol, ethanol, propanol and/or isopropanol, and/or water, and/or tetrahydrofuran. In some preferred embodiments, the first, second and/or third and/or subsequent liquid composition is formulated as a solution. The first, second and/or third and/or subsequent liquid composition may, optionally, contain additional ingredients, including further active agents, additives, or excipients. The first, second and/or third and/or subsequent liquid composition may, optionally, contain one or more binders, such as a polyamine, polyacrylate and/or polyurethane binder, as disclosed, for example, in US 2021/0156080; where the binders may be effective to improve adhesion of the coating to the coated surface. The one or more binders may comprise a polyurethane and/or an acrylic co-polymer emulsion and/or a polyurethane dispersion and/or a multifunctional acrylate, which is mixed or blended into the first, second and/or third and/or subsequent liquid composition. The first, second and/or third and/or subsequent liquid composition may, optionally, comprise a cross-linking agent as herein defined. The first, second and/or third and/or subsequent liquid composition may, optionally, comprise one or more additional anti-microbial agents, such as one or more additional cationic components, such as a quaternary ammonium salt, such as benzalkonium chloride and/or benzethonium chloride, optionally in an amount between about 0.001-1% w/v, optionally between about 0.005-1% w/v, for example between about 0.01-0.05% w/v.

The steps of applying said first liquid composition, said second liquid composition and/or said third and/or subsequent liquid composition to the substrate or article may, for example, each include (i) incubating the substrate or article in the respective liquid composition, and/or (ii) immersing the substrate or article in the respective liquid composition, and/or (iii) washing the substrate or article with the respective liquid composition, and/or (iv) dipping the substrate or article into the respective liquid composition either once or more than once, and/or (v) causing the respective liquid composition to flow over the substrate or article, and/or (vi) spraying the respective liquid composition onto the substrate or article, and/or (vii) painting the respective liquid composition onto the substrate or article, and/or (viii) wiping the respective liquid composition onto the substrate or article, and/or (ix) brushing the respective liquid composition onto the substrate or article, and/or (x) padding the liquid coating composition onto the substrate or article; and/or (xi) rolling the liquid coating composition onto the substrate or article, and/or (xii) applying the liquid coating composition onto the substrate or article by physical deposition, and/or (xiii) applying the liquid coating composition onto the substrate or article by electrophoretic deposition; or any combination or sequence of these application methods, which may be performed or repeated once or more than once.

Optionally, the method may further comprise an additional step of applying a binder composition to the substrate or article. The binder composition may, for example, comprise a polyamine, polyacrylate and/or polyurethane binder, as disclosed, for example, in US 2021/0156080; where the binder or binders may be effective to improve adhesion of the coating to the coated surface. The binder composition may be applied to the substrate or article in liquid form, for example as a solution, suspension, emulsion or dispersion. The binder composition may, in particular, comprise a polyurethane, and/or an acrylic co-polymer emulsion, and/or a polyurethane dispersion, and/or a multifunctional acrylate. The method of this aspect of the disclosure may accordingly include a step of applying a binder composition as disclosed to the substrate or article before or after any one of steps (a) to (d). The step of applying the binder composition to the substrate or article may comprise incubating the substrate or article in the binder composition, and/or immersing the substrate or article in the binder composition, and/or washing the substrate or article with the binder composition, and/or dipping the substrate or article into the binder composition either once or more than once, and/or causing the binder composition to flow over the substrate or article, and/or spraying the binder composition onto the substrate or article, and/or painting the binder composition onto the substrate or article, and/or wiping the binder composition onto the substrate or article, and/or brushing and/or padding and/or rolling the binder composition onto the substrate or article, and/or applying the binder composition to the substrate or article using any other suitable application technique or any combination of application techniques, including physical vapour deposition and/or electrophoretic deposition.

Optionally, the method may further comprise drying the coated substrate or article or allowing the coated substrate or article to dry, after application of each or any one layer and/or the binder composition, or after application of all the layers and/or the binder composition. The method may further comprise a step of heat-curing the coating, optionally by placing the substrate or article in an environment heated to a temperature of at least about 35° C. or at least about 50° C., for example to a temperature of about 140° C., preferably to a temperature of about 60-100° C. This may be particularly suitable where the substrate or article is not a part of a human or animal body, and/or is not formed from a natural or heat-sensitive material. Alternatively or additionally, the method may further comprise a step of UV-curing the coating in accordance with methods known in the art. For example. the UV version of guanidine GC (example 1b) can be cured using UV.

The present disclosure further embraces a substrate or article formed from an inert (non-living) material as herein defined, which substrate or article is coated with an anti-microbial coating according to the present disclosure.

An article or substrate that is coated with an anti-microbial coating according to the present disclosure may be prepared by coating the article or substrate with the anti-microbial coating in accordance with the methods of this disclosure. As elucidated above, these methods include applying the liquid coating composition of the disclosure to the surface of the article or substrate, either once or in multiple applications, such as two or more applications. The coating may be dried or cured between applications of the liquid coating composition or following all applications of the liquid coating composition. Alternatively, the coated substrate or article may be prepared using a “layering” approach, as elucidated above.

The present disclosure further embraces a method for preventing or reducing the growth or spread or the quantity of one or more micro-organisms on a substrate or article, and/or for inactivating one or more micro-organisms on a substrate or article, and/or for preventing the formation of and/or disrupting and/or removing a surface biofilm on a substrate or article, comprising the step of applying a coating to the substrate or article according to the methods of the present disclosure. The substrate or article may be formed from an inert (non-living) material, including porous and/or non-porous materials, and natural or artificial materials; or may be a body part of a living human or animal, such as the skin of a living human or animal.

The disclosure further embraces the use of a liquid coating composition in accordance with the disclosure for preventing or reducing the growth or spread or the quantity of one or more micro-organisms on a substrate or article, and/or for inactivating one or more micro-organisms on a substrate or article, and/or for preventing the formation of and/or disrupting and/or removing a surface biofilm on a substrate or article, whereby the liquid coating composition is applied to the substrate or article in accordance with the present disclosure. The substrate or article may be an inert (non-living) substrate or article, including porous and/or non-porous substrate or article, or a substrate or article made from natural or artificial materials; or may be a body part of a living human or animal.

The disclosure further embraces the use of a liquid coating composition in accordance with the disclosure in the manufacture of a composition which is suitable and effective for and/or for use in preventing or reducing the growth or spread or the quantity of one or more micro-organisms on a body part of a living human or animal and/or for inactivating one or more micro-organisms on a body part of a living human or animal.

The disclosure further provides a liquid coating composition as defined herein for use in a method of preventing or reducing the growth or spread or the quantity of one or more micro-organisms on a body part of a living human or animal, and/or for inactivating one or more micro-organisms on a body part of a living human or animal, such as the skin or hair of the human or animal; which method comprises applying the liquid coating composition to the body part; optionally, by washing and/or rinsing the body part with the liquid coating composition and/or by spraying, rubbing, padding, rolling, depositing and/or brushing the liquid coating composition onto the body part. The liquid coating composition may suitably be an antiseptic or sanitiser as herein defined. The method may suitably comprise applying the liquid coating composition to the hands of a human.

In one aspect, therefore, the disclosure provides an anti-microbial skin sanitiser product, such as a hand sanitiser product or a face sanitiser product, comprising a liquid coating composition in accordance with this disclosure, optionally formulated with one or more additional ingredients such as glycerol, moisturising agents, fragrances, and/or one or more additional anti-microbial agents, such as one or more additional cationic components, such as a quaternary ammonium salt, such as benzalkonium chloride and/or benzethonium chloride, optionally in a quantity of about 0.001-1% w/v, optionally about 0.005-1% w/v, or about 0.01-0.05% w/v, for use in a method of preventing or reducing the growth or spread or the quantity of one or more micro-organisms on the skin of a human and/or for inactivating one or more micro-organisms on the skin of a human; which method comprises applying the liquid coating composition to the skin, such as the face or hands, of the human by spraying or dispensing the composition onto the skin, such as the face or hands, of the human. Suitably, in these embodiments, the liquid coating composition may not comprise a guanidine compound. Suitably, in these embodiments, the total quantity of cationic polymers, including the alkyl urea polyalkylene imine and any additional cationic polymer(s), may not exceed 5% w/v.

In yet another aspect, the disclosure provides a method for preventing or reducing the growth or spread or the load or quantity of one or more micro-organisms on a surface, and/or for inactivating one or more micro-organisms on a surface, and/or for preventing the formation of and/or disrupting and/or removing a surface biofilm on a substrate or article, comprising the step of contacting the surface with a substrate or article that comprises a coating in accordance with this disclosure or that is coated in accordance with this disclosure. The coated substrate or article may be an item of cleaning equipment, such as a cloth or sponge. The coated substrate or article may be an item of personal protective equipment, such as gloves or a mask. The coated substrate or article may be a body part of a living human, such as the hands of a human. The contacted surface may be any surface that may be or may become contaminated with micro-organisms, including surfaces in healthcare settings, veterinary settings, dentistry settings, public settings, or private settings; and surfaces of medical devices and equipment, including personal protective equipment. The contacted surface may be an inert (that is, non-living) surface. In some embodiments, the contacted surface may be a living surface such as a part of the human or animal body, including human skin. This aspect of the disclosure is described herein as the “touch clean” effect and is described and demonstrated in the examples, where it is seen that coated substrates and articles in accordance with the disclosure are capable of disinfecting contaminated surfaces on contact.

In yet another aspect, the disclosure provides an alkyl urea polyalkylene imine polymer as herein disclosed for use in preventing or reducing the growth or spread or the load or quantity of one or more micro-organisms, including bacteria, viruses, fungi and/or yeast. The disclosure further embraces the use of an alkyl urea polyalkylene imine polymer as herein disclosed for preventing or reducing the growth or spread or the load or quantity of one or more micro-organisms, including bacteria, viruses, fungi, and/or yeast.

A method of inactivating or preventing or reducing the growth or spread or the quantity of one or more micro-organisms, in accordance with any aspect of this disclosure, may include inactivating or preventing or reducing the growth or spread or the quantity of bacteria. The bacteria may be bacteria that are associated with or are responsible for causing health care associated infections (HCAIs). In particular, the bacteria may be bacteria that are capable of forming a biofilm, particularly on a medical device or implant. The bacteria may be multi-drug resistant bacteria. In particular, the bacteria may be gram-positive or gram-negative bacteria; and may include any one or more of E. coli, S. aureus, E. hirae and P. aeruginosa strains. The bacteria may include antibiotic resistant bacteria, including MRSA and/or C. difficile.

A method of inactivating or preventing or reducing the growth or spread or the quantity of one or more micro-organisms, in accordance with any aspect of this disclosure, may include inactivating or preventing or reducing the growth or spread or the quantity of viruses. The viruses may be viruses that are associated with or are responsible for causing health care associated infections (HCAIs). The viruses may be enveloped and/or non-enveloped viruses; and may include adenovirus, norovirus, influenza virus, vaccinia virus and coronavirus strains, including but not limited to SARS-CoV-2.

A method of inactivating or preventing or reducing the growth or spread or the quantity of one or more micro-organisms, in accordance with any aspect of this disclosure, may include inactivating or preventing or reducing the growth or spread or the quantity of yeast and fungi and/or inactivating or preventing or reducing the growth or spread or the quantity of fungi or yeast, including but not limited to C. albicans strains. The yeast or fungi may be yeast or fungi that are associated with or are responsible for causing health care associated infections (HCAIs).

A method of preventing the formation of and/or disrupting and/or removing a surface biofilm on a substrate or article, in accordance with any aspect of this disclosure, may include disrupting, disturbing, removing, or preventing or inhibiting the surface formation or spread of any type of biofilm, particularly bacterial biofilms. The coatings and compositions of the present disclosure have proven efficacy against several microorganism which are known to be involved in the formation of pathogenic biofilms (Li et al, ibid). The cationic character of the disclosed coatings will also help to resist microbial adhesion, thus inhibiting the build-up of the microbial matrix structure of a biofilm.

As demonstrated in the following examples, the inventors have shown that the compositions of the present disclosure may have strong and durable anti-microbial effect against a wide range of different micro-organisms. The compositions may have long-term antimicrobial activity (antibacterial, antiviral, yeasticidal, and/or fungicidal) when applied to non-porous and porous surfaces, as well as skin with a long-term efficacy (180-365 days for surfaces and 48 hours for skin). The compositions have been shown to have a strong (at least log 4-99.99%) reduction effect against known nosocomial pathogens, including E. coli. S. aureus, MRSA, and Enterococcus, as well as influenza virus, norovirus, adenovirus, vaccinia and coronavirus strains such as SARS-CoV-2. This underpins the utility of the disclosed compositions for combatting HCAIs and common infections. The disclosed compositions have also been shown to be effective against MS2 bacteriophage, which is considered to be a surrogate for a non-enveloped virus, and against Phi6 bacteriophage which is considered to be a surrogate for an enveloped virus; further demonstrating the usefulness and efficacy of the disclosed compositions. The coatings are shown to have excellent characteristics, including stability, durability and lubricity, and can be effective to prevent the formation of or to disrupt or remove biofilms. The coatings disclosed herein are shown to be non-leaching and capable of resisting abrasion when applied to various surfaces or substrates including porous surfaces or substrates (fabrics) and non-porous surfaces or substrates (TPU), and as such are eminently suited for use on a wide range of substrates and applications. The coatings are also shown to be capable of being removed from skin by washing with soap and warm water, reinforcing their suitability for use as skin sanitisers. The “touch-clean” capability of the disclosed coatings, illustrated in the examples, provides further distinctive utility. The inventors have found that the disclosed coating compositions are capable of disinfecting any surface or skin to which they are applied; and that the unique ‘touch clean’ effect allows the technology to decontaminate any surface or person that it comes into contact with.

### SPECIFIC EXAMPLES

### Example 1: Synthesis of a Guanidine Compound (GC)

In a round-bottom flask, equipped with a condenser, a thermometer and a Pasteur pipette attachment to nitrogen inlet, 11.67 g of poly(ethylene glycol) methacrylate poly(hexanide) was blended and dissolved in 140.7 mL of water. 81 g (solid) of methoxy poly(ethylene glycol) methacrylate of MW 2000, purified on charcoal and diluted at 20% (w/v), was added with 16.21 g of methoxy poly(ethylene glycol) methacrylate of MW 350, 11.22 mL of methacrylic acid, 37.33 g of butyl methacrylate and 84.8 mL of isopropanol. The reflux condenser was turned on, the nitrogen allowed to bubble into the mixture of monomers and the heating turned up to warm up the mixture of monomers.

In a separate vial, 905 mg of potassium persulfate was dissolved in 24 mL of water and degassed with nitrogen.

Once the mixture in the round bottom flask had reached a temperature of 70° C., the potassium persulfate aqueous solution was added to the mixture of monomers in the round bottom flask and the polymerisation started.

The polymerisation was allowed to progress to the desired level of viscosity and was quenched by the addition of 100 mL of icy cold water. Once cooled down to room temperature, the polymerisation solution was dialysed at a molecular weight cut off of 12-14 KDa against water overnight.

### Example 1a: Synthesis of a Guanidine Compound (GC1 and GC2)

The polymer from example 1 in which the poly(ethylene glycol) methacrylate poly(hexanide) is replaced with poly(hexanide) methacrylate during the synthesis. The amount of poly(hexanide) methacrylate in the reaction is increased either to 2 fold (GC1) or 3 fold (GC2). The reaction and the work-up is carried out using the same procedure as in example 1.

### Example 1b: Synthesis of a Guanidine Compound (UV Active)

The polymer from example 1 in which methacrylic acid is replaced by 4-benzoylphenyl methacrylate (900 mg) during the synthesis, or in which both methacrylic acid and 4-benzoylphenyl methacrylate are jointly used.

### Example 2: Synthesis of alkyl(butyl, hexamethylene) urea polyethylene imine (bPEI)

Derivatisation of polyethylene imine (PEI) to alkyl urea polyethylene imine is carried out using an acylation reaction. PEI is reacted with n-butyl isocyanate or hexamethylene diisocyanate. The alkyl isocyanates are added drop-wise to the PEI. Butyl urea polyethylene imine and hexamethylene urea polyalkylene imine derivatives are commercially available from BioInteractions Ltd, Reading, United Kingdom.

### Example 3a: Production of a Liquid Coating Composition Containing PEI

Butyl urea PEI (bPEI) produced according to Example 2 was blended with polyethylene imine (PEI) and polyacrylic acid (PAA) in a mixture of IPA and water (approx. 70:30, IPA:water) in the following amounts:

| Components | Range of Concentration of components (%) |
|---|---|
| bPEI | 0.03-0.75 |
| PAA | 0.005-0.1 |
| PEI | 0.2-4.0 |
| Total concentration | 0.24-4.9 |

Benzalkonium chloride (0.005 to 0.05%) and glycerol (0.1-1%) was optionally added to the above formulations.

Compositions of varying strengths were prepared with C1 having a total concentration of 0.25%; C2 a total concentration of 2.1%; and C3 a total concentration of 4.2%

### Example 3b: Production of a Liquid Coating Composition Lacking PEI

Butyl urea PEI (bPEI) was blended with polyacrylic acid (PAA) in a mixture of IPA and water (approx. 70:30, IPA:water) in the following amounts

| Components | Range of Concentration of components (%) |
|---|---|
| bPEI | 0.2-4.5 |
| PAA | 0.005-0.1 |
| Total concentration | 0.205-4.6 |

Benzalkonium chloride and glycerol was optionally added to the above formulations.

Compositions of varying strengths were prepared with C4 having a total concentration of 0.25%; C5 a total concentration of 2.1%; and C6 a total concentration of 4.3%

### Example 4: Production of Liquid Coating Compositions Comprising Guanidine

Composition blends contain either three or four of the components. The range of concentrations for each component is shown in the tables below.

(i) Blend Composition Comprising of Component a (One of the Guanidine Compounds GC or GC1 or GC2), Component b (Butyl Urea Polyalkylene Imine bPEI), Component c (Polyacrylic Acid—PAA) and Component d (Polyethylene Imine-PEI)

| Components | Range of concentration of components (%) |
|---|---|
| bPEI | 0.01-7.0 |
| PAA | 0.002-0.1 |
| PEI | 0.05-4.0 |
| GC | 0.05-3.0 |
| GC1 | 0.5-3.5 |
| GC2 | 0.5-3.5 |

A cross linker and benzalkonium chloride were optionally added to the compositions.
D1 composition comprises of bPEI, PAA, PEI and GC, total concentration 4%
D2 composition comprises of bPEI, PAA, PEI and GC, total concentration 1.25%
D3 composition comprises of bPEI, PAA, PEI and GC1, total concentration 5%
D4 composition comprises of bPEI, PAA, PEI and GC2, total concentration 8%
   (ii) Blend Composition Comprising of Component a (One of the Guanidine Compounds —GC1 or GC2), Component b (Butyl Urea Polyalkylene Imine bPEI), and Component c (Polyacrylic Acid—PAA)

| Components | Range of concentration of components (%) |
|---|---|
| bPEI | 0.3-4.5 |
| PAA | 0.009-0.05 |
| GC1 | 0.3-3.0 |
| GC2 | 0.1-3.0 |

A cross linker and benzalkonium chloride were optionally added to the compositions.
D5 composition comprises of bPEI, PAA and GC1, total concentration 4.0%
D6 composition comprises of bPEI, PAA and GC2, total concentration 4.5%

### Example 5: Coating Methods

The compositions of Examples 3 and 4 were used to coat substrates of various kinds, including TPU strips, fabrics, gloves and hands, by spraying or brushing or painting the compositions onto the substrates, or by dipping the substrates into the compositions. The substrates were allowed to dry.

Surfaces including TPU strips and fabrics were also coated using a layering method. A first layer was applied to the surface by applying an aqueous-alcoholic coating solution containing 0.05-7% w/v of butyl urea polyalkylene imine and/or PAA (0.002-0.1%). The first layer was allowed to dry, prior to application of a second layer, which was done by applying an aqueous-alcoholic coating solution containing 0.05-7% w/v of butyl urea polyalkylene imine, 0.3-8% w/v of polyethylene imine (PEI) and 0.5-3.5% w/w of guanidine compound and and/or PAA (0.002-0.1%) The coated substrate was then allowed to dry.

### Example 6: Methodologies for Antimicrobial Testing

Formulations of the disclosure were tested for efficacy in the following tests:

### EN 14476 Antiviral Suspension Test

This antiviral test was carried using the method based on EN 14476 (Quantitative suspension test for the evaluation of virucidal activity; pass criteria 4-log) where the hand sanitizer solution is tested against a viral suspension. For this test, the hand sanitizer is made to ×1.25 the concentration of the desired formulation as it is diluted during the addition of the virus inoculum and interference substance. The antiviral activity is determined by comparing the log reduction against a negative control. Virus inoculums were made to a concentration of 10⁸ PFU/ml in the relevant media for the specific virus. A virus suspension is made by adding 1 ml of the virus inoculum to 1 ml of bovine serum (0.3 g/L) for clean conditions and 3 ml/L sheep erythrocytes for dirty conditions. This suspension is added to 8 ml of the x1.25 concentration of the hand sanitizer solution and vortex mixed. The mixture is left at a controlled temperature (e.g. 20° C.) for the desired contact time (i.e. 1 minute and 2 minutes). Post contact time serial dilutions are made and quantified for both the hand sanitiser solution and the negative control.

### EN 13727 Antibacterial Suspension Test

This antibacterial test was carried using the method based on EN 13727 (Quantitative suspension test for the evaluation of bacterial activity; pass criteria 5-log) where the hand sanitizer solution is tested against a bacterial suspension. For this test, the hand sanitizer is made to ×1.25 the concentration of the desired formulation as it is diluted during the addition of the bacterial inoculum and interference substance. The antibacterial activity is determined by comparing the log reduction against a negative control. Bacterial inoculums were made to a concentration of 10⁸ PFU/ml in the relevant media for the specific bacteria. A bacterial suspension is made by adding 1 ml of the virus inoculum to 1 ml of bovine serum (0.3 g/L) for clean conditions and 3 ml/L sheep erythrocytes for dirty conditions. This suspension is added to 8 ml of the x1.25 concentration of the hand sanitizer solution and vortex mixed. The mixture is left at a controlled temperature (e.g. 20° C.) for the desired contact time (i.e. 1 minute and 2 minutes). Post contact time serial dilutions are made and quantified for both the hand sanitiser solution and the negative control.

### EN 13624 Yeasticidal Suspension Test

This yeasticidal test was carried using the method based on EN 13624 (Quantitative suspension test for the evaluation of yeasticidal activity; pass criteria 4-log) where the hand sanitizer solution is tested against a yeast suspension. For this test, the hand sanitizer is made to ×1.25 the concentration of the desired formulation as it is diluted during the addition of the yeast inoculum and interference substance. The yeasticidal activity is determined by comparing the log reduction against a negative control. Yeast inoculum was made to a concentration of 10⁷ PFU/ml in the relevant media. A yeast suspension is made by adding 1 ml of the yeast inoculum to 1 ml of bovine serum (0.3 g/L) for clean conditions and 3 ml/L sheep erythrocytes for dirty conditions. This suspension is added to 8 ml of the x1.25 concentration of the hand sanitizer solution and vortex mixed. The mixture is left at a controlled temperature (e.g. 20° C.) for the desired contact time (i.e. 1 minute and 2 minutes). Post contact time serial dilutions are made and quantified for both the hand sanitiser solution and the negative control.

### ISO 20743 General Method (Antibacterial Activity on Porous Material)

The antimicrobial testing on porous material is based on ISO 20743 (Textiles Determination of antibacterial activity of textile products) using the absorption method, where the bacterial inoculum is directly placed onto the tested surface. The bacterial inoculum is made to a concentration of 10⁵ CFU/ml in tryptone soya broth (TSB) and applied to the textiles that have no antibacterial activity (uncoated) and treated textiles (coated). After inoculation, the treated and the control samples are incubated at 37° C. for the desired contact time. Saline is then used to recover the bacterial cells from the porous surface. Post contact time serial dilutions are made, and the bacteria are quantified. The log reduction is determined by comparing the uncoated surface with the coated surface.

### ISO 22196 General Method (Antibacterial Activity on Non-Porous Material)

The antimicrobial testing on non-porous material is based on ISO 22196 (Measurement of antibacterial activity on plastics and other non-porous surfaces). The samples are placed within a petri dish where the bacterial inoculum at a concentration of 10⁶ CFU/ml in 0.2% nutrient broth is directly placed onto the surface and covered with a slip with a known surface area. After inoculation, the treated and the control samples are incubated at 37° C. for the desired contact time. TSB is used to recover the bacterial cells from the material. Post contact time serial dilutions are made, and the bacteria are quantified. The log reduction is determined by comparing the uncoated surface with the coated surface.

### ISO 18184 General Method (Antiviral Activity on Porous Material)

The antiviral testing on porous material is based on ISO 18184 (Textiles Determination of antiviral activity of textile products) using the absorption method, where the virus inoculum at a concentration of 10⁷ PFU/ml in a relevant media is directly placed onto the surfaces to be tested; uncoated textiles and treated textiles (coated). The treated and the control samples are incubated at a controlled temperature for the desired contact time. The relevant media is then used to recover the viruses from the porous surface of the material. Post contact time serial dilutions are made, and the viruses are quantified. The log reduction is determined by comparing the uncoated surface with the coated surface.

### ISO 21702 General Method (Antiviral Activity on Non-Porous Material)

The antiviral testing on non-porous material is based on ISO 21702 (Measurement of antiviral activity on plastics and other non-porous surfaces). The samples are placed within a petri dish where the virus inoculum at a concentration of 10⁷ PFU/ml in a relevant media is directly placed onto the surface and covered with a slip with a known surface area. The treated and the control samples are incubated at a controlled temperature for the desired contact time. The relevant media is then used to recover the viruses from the surface of the samples, serial dilutions are made, and the viruses are quantified. The log reduction is determined by comparing the uncoated surface with the coated surface.

### Bacterial Sneeze Test

This test is for determining the antimicrobial activity of treated surfaces (non-porous and porous) when sprayed with bacterial suspension in order to mimic a sneeze from an individual onto a surface. For this test, the preparation of the samples, the harvesting of the cells and the quantification of the antimicrobial activity was carried out in the same way as for the antimicrobial test on non-porous and porous surfaces (ISO 20743+ISO 22196). The difference in this test was that both treated and untreated samples were sprayed with the bacterial suspension, in order to mimic a sneeze. The non-porous samples were not covered with a cover slip as the suspension of the bacteria adhered to the surface.

### EN 1500 Method

The EN 1500 test is to determine the efficacy of the hand sanitizer formulation when applied onto the hands of a volunteer. The EN 1500 method consists of carrying out a wash method which is performed prior to each of the inoculation processes. The inoculation of the bacteria is carried out on untreated hands, treated hands with the reference product (i.e. IPA) and treated hands with the test product. The antibacterial activity is determined by comparing the number of bacteria recovered from the untreated hands and treated hands. The product must have an equal or greater antibacterial activity than the reference product used.

### (i) Hand Wash Method

The wash method from EN 1500 consists of the hands of the volunteer to be first rubbed with 5 ml of diluted soap for 1 minute to sterilise the hands. After rubbing with the diluted soap, the hands are rinsed with water and then dried with a paper towel for 30 seconds to remove any diluted soap.

### (ii) Inoculation Process

The inoculation method consists of the following procedure. The bacteria (E. coli: 10⁸ cfu/ml) in TSB is poured into a container where both hands are immersed up to the mid-metacarpals for 5 seconds with fingers spread apart. The hands are then removed from the inoculation liquid and the excess liquid is allowed to drain back into the container for a maximum of 30 seconds. The hands are then dried in the air for 3 min whilst holding them in a horizontal position with the fingers spread apart to avoid formulation of droplets. Each of the hands are then placed in a separate sterile petri dish containing 10 ml of TSB whilst rubbing the bottom of the petri dish for 1 minute. The hands are then washed again using the same method as described as above.

(iii) Hands Treated with Reference Product or Test Product:

Before inoculation, the hands are either treated with a reference product (IPA) or testing product (i.e. hand sanitizer). This involves pouring 3 ml of the product into cupped dry hands and rubbing vigorously for 30 seconds using the hand rub procedure set out in the standard. This process is carried out twice, therefore a total rubbing time of 1 minute using 6 ml of the product. After applying the product, the hands are inoculated with the bacteria using the inoculation process above.

The treated hands go through the same inoculation procedure except instead of rubbing the finger in 10 ml of TSB, the hands are rubbed in 10 ml of neutralizing fluid. Serial dilutions of each sampling fluid for both the untreated hands and treated hands are quantified. The antibacterial activity is determined by comparing the results from the untreated and treated hands of the volunteers.

### Pig Skin Study Over 48 Hours

The efficacy of the hand sanitizer on pig skin was tested over days (Day 0,1,2) against bacteriophage Phi6. For this test untreated pig skin samples were compared against pig skin samples that were treated with the various hand sanitizer formulations. Pig skin was cut into sample size and a set number of pig skin samples were sprayed with the various hand sanitizer formulations. After applying the hand sanitizer formulations, the untreated and treated samples were tested at Day 0, 1 and 2. For testing the antiviral activity, both the untreated and treated pig skin samples were sprayed with 10⁷ PFU/ml of bacteriophage Phi6 and left for contact time of 5 minutes and 60 minutes. After the desired contact times, the pig skin samples were transferred to TSB and vortexed. The number of bacteriophages on the pig skin samples were quantified. The antiviral activity was determined by comparing the number of bacteriophages from the untreated and the treated pig samples.

### EN 13697: Bacterial and Yeasticidal Non-Porous Solution Test

This test is a surface solution test based on EN 13697 (Quantitative nonporous surface test for the evaluation of bactericidal and/or fungicidal activity; pass criteria 4-log) where the hand solution is tested against a pathogen that has been dried on a stainless steel disc. The antimicrobial activity is determined by comparing the log reduction against a negative control (hard water). The bacterial/yeast inoculums were made to a concentration of 10⁷-10⁸ CFU/ml in the relevant media for the specific pathogen. A bacterial/yeast suspension is made by adding 1 ml of the inoculum to 1 ml of bovine serum (0.3 g/L) for clean conditions and 3 ml/L sheep erythrocytes for dirty conditions. A stainless steel disc is placed inside a sterile petri dishes and 50 μl of the microbial test suspension is pipetted on top. The suspension in the dried at 37° C. until the inoculum is visible dry. After drying the 100 μl of the hand solution is pipetted on top of the dried inoculum and left for the desired contact time (1 minute to 60 minutes). At the end of the contact time the stainless-steel disc is transferred into 10 ml of neutralising media and serial dilutions are made to quantify the number of microorganisms remaining on the surface of the stainless-steel disc.

### EN 16777: Antiviral Activity Non-Porous Solution Test

This test is a surface solution test based on EN 16777 (Quantitative nonporous surface test for the evaluation of virucidal activity; pass criteria 4-log) where the hand solution is tested against a virus suspension that has been dried on a stainless-steel disc. The antiviral activity is determined by comparing the log reduction against a negative control (hard water). The viral inoculums were made to a concentration of 10⁸ PFU/ml in the relevant media. A viral suspension is made by adding 1 ml of the inoculum to 1 ml of bovine serum (0.3 g/L) for clean conditions and 3 ml/L sheep erythrocytes for dirty conditions. A stainless-steel disc is placed inside a sterile petri dish and 50 μl of the viral test suspension is pipetted on top. The suspension in the dried at 37° C. until the inoculum is visible dry. After drying the 100 μl of the hand solution is pipetted on top of the dried inoculum and left for the desired contact time (1 minute to 60 minutes). At the end of the contact time the stainless-steel disc is transferred into 10 ml of neutralising media and serial dilutions are made to quantify the number of microorganisms remaining on the surface of the stainless-steel disc.

### EN 14561: Bactericidal Solution Carrier Test on Medical Instruments.

This test is a surface solution test based on EN 14561 (Quantitative carrier test for the evaluation of bactericidal activity for instruments used in the medical area; pass criteria 5-log) where the hand solution is tested against a bacterial suspension that has been dried on a glass carrier. The antimicrobial activity is determined by comparing the log reduction against a negative control (hard water). The bacterial inoculum is made to a concentration of 10⁹ CFU/ml in the relevant media for the specific pathogen. A bacterial suspension is made by adding 9 ml of the inoculum to 1 ml of bovine serum (0.3 g/L) for clean conditions and 3 ml/L sheep erythrocytes for dirty conditions. The bacterial suspension is mixed and 50 μl is pipetted on the “inoculation square” of a carrier and distribute equally inside the square, with the tip of the pipette. The suspension in the dried at 37° C. until the inoculum is visible dry. After drying the contaminated glass side is immersed in a sample of the hand solution/hard water and left for the desired contact time. After the contact time the carrier is then transferred into a neutralising media and vortex mixed to remove the bacteria from the surface. Serial dilutions of this sampling fluid are then made, and the number of bacteria is quantified.

### EN 14562: Yeasticidal Solution Carrier Test on Medical Instruments.

This test is a surface solution test based on EN 14562 (Quantitative carrier test for the evaluation of yeasticidal activity for instruments used in the medical area; pass criteria 5-log) where the hand solution is tested against a yeast suspension that has been dried on a glass carrier. The antimicrobial activity is determined by comparing the log reduction against a negative control (hard water). The yeast inoculum is made to a concentration of 10⁹ CFU/ml in the relevant media for the specific pathogen. A bacterial suspension is made by adding 9 ml of the inoculum to 1 ml of bovine serum (0.3 g/L) for clean conditions and 3 ml/L sheep erythrocytes for dirty conditions. The bacterial suspension is mixed and 50 μl is pipetted on the “inoculation square” of a carrier and distribute equally inside the square, with the tip of the pipette. The suspension in the dried at 37° C. until the inoculum is visible dry. After drying the contaminated glass side is immersed in a sample of the hand solution/hard water and left for the desired contact time. After the contact time the carrier is then transferred into a neutralising media and vortex mixed to remove the bacteria from the surface. Serial dilutions of this sampling fluid are then made, and the number of bacteria is quantified.

Modified EN 13697 with EN 1500 (Treated Hand Disinfecting a Surface)

The modified EN 13697 (Surface disinfectant test) with EN 1500 procedure was used to determine the efficacy of the hand sanitiser when a treated hand touches an inoculated stainless-steel disk (i.e. demonstrating that the treated hand is disinfecting a contaminated surface). For this procedure, the number of bacteria on the stainless-steel disc and on the hands of the volunteer after touching the surface was quantified. To determine the ability of the treated hands to disinfect a contaminated surface, comparison of the number of bacteria on the inoculated surface and the untreated/treated hands was performed.

Modifed EN 13697 (Bacteria)+Modified EN 16777 (Virus) with Treated Gloves

The modified EN 13697 (antibacterial surface disinfectant test EN 16777 (antiviral surface disinfectant test) procedure was used to determine the efficacy of a coated glove when it touches an inoculated stainless-steel disk (i.e. demonstrating that the treated glove is disinfecting a contaminated surface).

For this procedure, the number of microorganisms (bacteria/bacteriophage) on the stainless-steel disc and on the treated glove after touching the surface was quantified. To determine the ability of the treated glove to disinfect a contaminated surface, comparison of the number of microorganisms on the inoculated surface and the untreated/treated gloves was performed.

### (i) EN 13697 Inoculation of Stainless-Steel Discs

The following procedure was used to inoculate the discs. One stainless steel disc was used for each hand or glove. Each stainless-steel disc was inoculated with 10⁸ CFU/ml of bacteria and dried by placing the disk in an oven.

### (ii) EN 1500 Hand Wash Method

The hands of the volunteer were first rubbed with 5 ml of diluted soap for 1 minute to sterilise the hands. After rubbing with diluted soap, the hands were rinsed with water and dried with paper towel for 30 seconds to remove any remaining soap.

(iii) Hands Treated with Test Product:

The hands were sprayed with the formulation and left to air dry to ensure that no liquid remains on the surface of each hand.

### (iv) Hands or Gloves Touching Contaminated Surface Procedure

This method was carried out on both the untreated and treated hands or gloves. Each hand or gloved hand was pressed down on top of the inoculated discs. The hands or gloves were then placed in a separate sterile petri dish containing the relevant media whilst rubbing the bottom of the petri dish. The touched inoculated stainless-steel discs were transferred to vials containing the relevant media and then vortexed. Serial dilutions of the sampling fluid from the stainless-steel discs and the sampling fluid for the hands or gloves were prepared and quantified. The only difference for the treated hand or glove was that the sampling fluid used was a neutralising fluid rather than media.

### Example 7: Antimicrobial Test Results

### EN 14476: Antiviral Suspension Test

The EN 14476 test was carried out against bacteriophage Phi6 on the hand sanitiser solution. Compositions C1-C6 were tested and showed a high level of antiviral activity against bacteriophage Phi6.

TABLE 1
EN 14476 Virucidal: Antiviral suspension test composition
C1-C3 against bacteriophage Phi6 at 1 min contact time.

| Standard | Formulation | Log reduction |
|---|---|---|
| EN 14476 | C1 | >7.45 |
| | C2 | >7.45 |
| | C3 | >7.45 |

TABLE 2
EN 14476 Virucidal: Antiviral suspension test composition
C5-C6 against bacteriophage Phi6 at 1 min contact time.

| Standard | Formulation | Log reduction |
|---|---|---|
| EN 14476 | C4 | >7.45 |
| | C5 | >7.45 |
| | C6 | >7.45 |

The EN 14476 test was carried out on compositions according to the present disclosure against a number of viruses as listed below. The results showed that the disclosed compositions display a high level of antiviral activity against both non-enveloped and enveloped viruses including a surrogate for SARS-CoV-2 (human coronavirus 229e).

TABLE 3
Virucidal EN 14476: Summary of the log reductions
of the quantitative suspension test.

| Virus | Log reduction |
|---|---|
| Adenovirus | >4.63 |
| Murine norovirus | >5.32 |
| Vaccinia virus | >5.19 |
| Human coronavirus | >4.00 |
| Influenza | >4.51 |
| SARS-CoV-2 | >4.00 |

### EN 13727: Antibacterial Suspension Test

The EN 13727 test was carried out on compositions according to the present disclosure against a number of bacteria as listed below. The results showed a high level of antibacterial activity against both gram-negative and gram-positive bacteria.

TABLE 4
Bactericidal EN 13727: Summary of the log reductions
of the quantitative suspension test.

| Bacteria | Log reduction |
|---|---|
| Enterococcus hirae | >5.47 |
| Escherichia coli | >5.37 |
| Pseudomonas aeruginosa | >5.52 |
| Staphylococcus aureus | >5.14 |
| Methicillin-resistant staphylococcus aureus | >5.51 |
| subsp. aureus (MRSA) | |

### EN 13624: Yeasticidal Suspension Test

The EN 13624 test was carried out on compositions according to the present disclosure against Candida albicans on a hand sanitiser solution according to the present disclosure.

A high level of yeasticidal activity was achieved.

TABLE 5
Yeasticidal EN 13624: Log reduction
of the quantitative suspension test.

| Yeast | Log reduction |
|---|---|
| Candida albicans | >4.52 |

### EN 13697: Bacterial and Yeasticidal Non-Porous Solution Test

The EN 13697 test was carried out against both gram-negative and gram-positive bacteria and yeast on a disinfectant composition according to the present disclosure. A high level of antimicrobial activity was achieved on a non-porous surface (i.e. stainless steel discs).

TABLE 6
Bactericidal and Yeasticidal EN 13697: Summary of the log
reductions of the quantitative non-porous solution test.

| Bacteria and Yeast | Log reduction |
|---|---|
| Enterococcus hirae | >4.58 |
| Escherichia coli | >6.62 |
| Pseudomonas aeruginosa | >6.17 |
| Staphylococcus aureus | >5.02 |
| Candida albicans | >5.96 |

### EN 16777: Antiviral Activity Non-Porous Solution Test

The EN 16777 test was carried out against both non-enveloped and enveloped viruses on a disinfectant composition according to the present disclosure. A high level of antiviral activity was achieved on both types of viruses on a non-porous surface (i.e. stainless steel discs).

TABLE 7
Virucidal EN 16777: Summary of the log reductions
of the quantitative non-porous solution test.

| Virus | Log reduction |
|---|---|
| Adenovirus | >4.0 |
| Murine norovirus | >4.0 |
| Vaccinia virus | >4.0 |

### EN 14561: Bactericidal Solution Carrier Test on Medical Instruments.

The EN 14561 test was carried out against both gram-negative and gram-positive bacteria on a disinfectant composition according to the present disclosure. A high level of antibacterial activity was achieved on a non-porous surface (i.e. glass).

TABLE 8
Bactericidal EN 14561: Summary of the log reduction
of the quantitative carrier test for instruments.

| Bacteria | Log reduction |
|---|---|
| Enterococcus hirae | >5.21 |
| Pseudomonas aeruginosa | >5.27 |
| Staphylococcus aureus | >6.02 |

### EN 14562: Yeasticidal Solution Carrier Test on Medical Instruments.

The EN 14562 test was carried out against Candida albicans on a disinfectant composition according to the present disclosure. Yeasticidal activity was achieved on a non-porous surface (i.e. glass).

TABLE 9
Yeasticidal EN 14562: Log reduction of the
quantitative carrier test for instruments.

| Yeast | Log reduction |
|---|---|
| Candida albicans | >4.33 |

### Bactericidal EN 1500: Hygienic Hand Rub

The EN 1500 test was carried out to determine the efficacy of a hand sanitizer formulation according to this disclosure when applied onto the hands of volunteers. Results from this study showed that the sanitiser formulation was efficacious and passed the criteria of the standard.

TABLE 10
Bactericidal EN 1500: log reduction
of the hand hygiene hand rub test.

| Bacteria | Log reduction |
|---|---|
| Escherichia coli | >4.15 |

### Pig Skin Study Over 48 Hours Results Against Bacteriophage Phi6:

The efficacy of the hand sanitizer on pig skin was tested over 2 days (day 0, 1, 2) against bacteriophage Phi6. The results show that the sanitiser formulation of the current disclosure is still present over the 48 h time period and maintains its efficacy.

TABLE 11
Pig skin study: Summary of log reductions
of the hand sanitiser over 48 h.

| | Day 0 log | | Day 1 log | | Day 2 log | |
|---|---|---|---|---|---|---|
| | reduction | | reduction | | reduction | |
| Formulation | 5 min | 60 min | 5 min | 60 min | 5 min | 60 min |
| C2 | 2.2 | 3.7 | 2.2 | 2.5 | 3.3 | 3.6 |
| C4 | 3.5 | 3.6 | 3.3 | 3.5 | 3.2 | 3.5 |

### Surface Results

### ISO 20743: Antibacterial Activity on Porous Material

Formulations C1-C6 were applied to polyester cellulose. The antibacterial activity against E. coli is shown below. The results show that the various compositions at similar concentrations had similar antibacterial activity.

TABLE 12
ISO 20743 antibacterial activity: Summary of
the log reductions on polyester cellulose.

| Formulation | Log reduction |
|---|---|
| C1 | >1.6 |
| C2 | >1.8 |
| C3 | >3.2 |
| C4 | >1.6 |
| C5 | >2.2 |
| C6 | >4.7 |

### ISO 22196: Antibacterial Activity on Non-Porous Material

Formulations C1-C6 were applied to polyurethane squares. The antibacterial activity against E. coli are shown below.

TABLE 13
ISO 22196 antibacterial activity: Summary of
the log reductions on polyurethane squares.

| Formulation | Log reduction |
|---|---|
| C1 | >3.3 |
| C2 | >4.8 |
| C3 | >5.8 |
| C4 | >3.4 |
| C5 | >4.8 |
| C6 | >5.8 |

### ISO 18184: Antiviral Activity on Porous Material

Formulations C1-C6 were applied to polyester cellulose. The antiviral activity against bacteriophage Phi6 are shown below. Antiviral activity was achieved across all concentrations.

TABLE 14
ISO 18184 antiviral activity: Summary of the
log reductions on polyester cellulose.

| Composition tested | Log reduction |
|---|---|
| C1 | >4.7 |
| C2 | >4.7 |
| C3 | >4.7 |
| C4 | >3.5 |
| C5 | >4.7 |
| C6 | >4.7 |

### ISO 21702: Antiviral Activity on Non-Porous Material

Formulations C1, C2, and C4-C5 were applied on polyurethane squares. The antiviral activity against bacteriophage Phi6 are shown below in Table 15.

TABLE 15
ISO 21702 antiviral activity: Summary of the
log reductions on polyurethane squares.

| Composition tested | Log reduction |
|---|---|
| C1 | >0.7 |
| C2 | >3.8 |
| C4 | >0.8 |
| C5 | >4.0 |

### ISO 20743: Antibacterial Activity on a Porous Surface Post Exposure to a Bacterial Sneeze

The antibacterial activity of formulation C2 on polyester cellulose sheet when exposed to a sneeze with a bacterial load of approximately 10⁶ cfu/ml. Samples were coated and tested on the same day or left for several days before being subjected to the bacterial sneeze test. The results show that the coating is effective on a porous surface when exposed to a bacterial sneeze and has antibacterial activity against both gram-negative and gram-positive bacteria.

TABLE 16
ISO 20743 antibacterial activity: Summary of the log reductions
on polyester cellulose exposed to a bacterial sneeze.

| Treated surface age | Microorganism | Log reduction |
|---|---|---|
| Day 0 | E. coli | >8.0 |
| | S. aureus | >6.2 |
| Day 6 (real time) | S. aureus | >7.0 |

### ISO 22196: Antibacterial Activity on a Non-Porous Surface Post Exposure to a Bacterial Sneeze

The antibacterial activity of formulation C2 on polyurethane squares when exposed to a sneeze with a bacterial load of approximately 10⁶ cfu/ml. Samples were coated and tested on the same day or left for several days before being subjected to the bacterial sneeze test. The results show that the coating is effective on a non-porous surface when exposed to a bacterial sneeze and has antibacterial activity against both gram-negative and gram-positive bacteria.

TABLE 17
ISO 22196 antibacterial activity: Summary of the log reductions
on polyurethane squares exposed to a bacterial sneeze.

| Treated surface age | Microorganism | Log reduction |
|---|---|---|
| Day 0 | E. coli | >7.7 |
| | S. aureus | >7.3 |
| Day 6 | E. coli | >7.9 |
| | S. aureus | >6.8 |

### Antibacterial Activity on Porous and Non-Porous Material (ISO 20743 22196)

The antibacterial activity of Composition D1 against E. coli on porous (e.g. polyester cellulose sheet, masks) and non-porous (e.g. thermoplastic polyurethane (TPU)) surfaces are shown in Table 18. The results show that a high level of antibacterial activity was obtained on different porous and non-porous materials.

TABLE 18
Antibacterial activity of Composition D1 against
E. Coli porous and non-porous materials.

| | Porous (polyester | | Non-Porous |
|---|---|---|---|
| Antibacterial | cellulose) log | Porous (mask) | (TPU) log |
| activity | reduction | log reduction | reduction |
| ISO 20743 | >5.6 | >5.6 | — |
| ISO 22196 | — | — | >6.2 |

### Antibacterial Activity on Porous and Non-Porous Material (ISO 20743 22196)

The antibacterial activity of Composition D6 against E. coli on porous (e.g. polyester cellulose) and non-porous (e.g. TPU)) are shown in Table 19. The results show that a high level of antibacterial activity was obtained on both porous and non-porous materials.

TABLE 19
Antibacterial activity of Composition D6 against
E. Coli porous and non-porous materials.

| | Porous (polyester | Non-Porous |
|---|---|---|
| Antibacterial | cellulose) log | (TPU) log |
| activity | reduction | reduction |
| ISO 20743 | >4.4 | — |
| ISO 22196 | — | >6.2 |

Antibacterial Activity on Porous Material that Underwent Aging (ISO 20743)

The antibacterial activity against E. coli and S. aureus on polyester cellulose sheet samples are shown in Table 20. Samples were coated with composition D1 and underwent aging and then were tested for antibacterial activity. The results show that the coated samples maintained a high level of antibacterial activity over twelve-months on a porous surface. Additionally, the coating is effective against both gram-negative and gram-positive bacteria.

TABLE 20
Summary of antibacterial results against E. Coli and
S. aureus on polyester cellulose sheet aged samples.

| | | |
|---|---|---|
| Treated surface age | Microorganism | Log reduction |

| | | | |
|---|---|---|---|
| Day | 0 | E. coli | >7.4 |
| Six | months | E. coli | >8.3 |
| | | S. aureus | >7.1 |
| Twelve | months | S. aureus | >7.7 |

### Antibacterial Activity on a Porous Surface Post Exposure to a Bacterial Sneeze (ISO 20743)

This test is for determining the antibacterial activity of a coated porous surface when sprayed with a bacterial suspension (E. coli and S. aureus) to mimic sneezing from an individual onto the surface.

The antibacterial activity of Composition D1 on polyester cellulose sheet when exposed to a sneeze with a bacterial load of approximately 10⁴-10⁶ cfu/ml. Samples were coated, subjected to the bacterial sneeze and tested on the same day.

The results in Table 21 show that the coating is effective on a porous surface when exposed to a bacterial sneeze and has antibacterial activity against both gram-negative and gram-positive bacteria.

TABLE 21
Antibacterial activity of Composition D1 against E. Coli and
S. Aureas on polyester cellulose sheet when exposed to a sneeze.

| Microorganism | Log reduction |
|---|---|
| E. coli | >7.1 |
| S. aureus | >6.1 |

Antibacterial Activity on Non-Porous Material that Underwent Aging (ISO 22196

The antibacterial activity against E. coli and S. aureus on polyurethane squares is shown in Table 22. Samples were coated with composition D1 and underwent aging and then were tested for antibacterial activity.

The results show that the coated samples maintained a high level of antibacterial activity over twelve-months on a non-porous surface. Additionally, the coating is effective against both gram-negative and gram-positive bacteria.

TABLE 22
Summary of antibacterial results against E. Coli and
S. aureus on polyester cellulose sheet aged samples.

| | | |
|---|---|---|
| Treated surface age | Microorganism | Log reduction |

| | | | |
|---|---|---|---|
| Day | 0 | E. coli | >6.7 |
| | | S. aureus | >5.9 |
| Six | months | E. coli | >4.0 |
| | | S. aureus | >5.1 |
| Twelve | months | E. coli | >6.1 |
| | | S. aureus | >4.8 |

### Antibacterial Activity on a Non-Porous Surface Post Exposure to a Bacterial Sneeze (ISO 22196)

This test is for determining the antibacterial activity of a coated non-porous surface when sprayed with a bacterial suspension (E. coli and S. aureus) to mimic sneezing from an individual onto the surface.

The antibacterial activity of Composition D1 on polyurethane squares was tested when exposed to a sneeze with a bacterial load of approximately 10⁴-10⁶ cfu/ml. Samples were coated and either tested on the same day or left for several days before being subjected to the bacterial sneeze test.

The results in Table 23 show that the coating is effective on a non-porous surface when exposed to a bacterial sneeze and has antibacterial activity against both gram-negative and gram-positive bacteria.

TABLE 23
Antibacterial activity of Composition D1 against E. Coli and
S. Aureus on polyester cellulose sheet when exposed to a sneeze.

| Treated surface age | Microorganism | Log reduction |
|---|---|---|
| Day 0 | E. coli | >7.2 |
| | S. aureus | >7.5 |
| Day 5 | E. coli | >7.9 |
| Day 6 | S. aureus | >6.8 |

### Yeasticidal Activity of Composition D1 on TPU (ISO 22196)

The yeasticidal activity of Composition D1 against Candida albicans on TPU samples was determined. The samples were coated and then inoculated with Candida albicans. Results are presented in Table 24 showing yeasticidal activity was achieved.

TABLE 24
Yeasticidal activity against Candida albicans on TPU.

| Composition | Log reduction |
|---|---|
| D1 | >4.8 |

### Antiviral Activity on Porous Material (ISO 18184)

The antiviral activity of different compositions against Bacteriophage Phi6 (enveloped) and MS2 (non-enveloped) on porous surface (e.g. polyester cellulose) was determined using the ISO 18184 test. The results are shown in Table 25. The antiviral activity was also determined against Vaccinia virus (enveloped), Adenovirus (non-enveloped), Influenza (enveloped) on masks as shown in Table 26. The results showed that antiviral activity was achieved on porous surfaces against non-enveloped and enveloped bacteriophages and non-enveloped and enveloped viruses.

TABLE 25
Antiviral activity against Bacteriophages
Phi6 and MS2 on polyester cellulose.

| | Bacteriophage Phi6 | Bacteriophage MS2 |
|---|---|---|
| Composition | Log reduction | Log reduction |
| D1 | >4.6 | >2.6 |
| D2 | >4.6 | n/a |
| D5 | >5.0 | >2.6 |
| D6 | >4.6 | n/a |

TABLE 26
Antiviral activity against Vaccinia virus,
Adenovirus, Influenza on masks.

| Virus | Log reduction |
|---|---|
| Adenovirus | >4.0 |
| Vaccinia virus | >4.13 |
| Influenza | >5.42 |

### ISO 21702: Antiviral Activity on Non-Porous Material

The antiviral activity of different compositions against Bacteriophage Phi6 and MS2 on non-porous surface (e.g. TPU) was determined using the ISO 21702 test. The results are shown in Table 27. The antiviral activity of the composition against Influenza on TPU is shown in Table 28. Antiviral activity was achieved on a non-porous surface against non-enveloped and enveloped bacteriophages and enveloped virus.

TABLE 27
Antiviral activity against Bacteriophage Phi6 and MS2 on TPU.

| | Bacteriophage Phi6 | Bacteriophage MS2 |
|---|---|---|
| Composition | Log reduction | Log reduction |
| D1 | >4.8 | >0.8 |
| D2 | >2.0 | n/a |
| D3 | n/a | >1.2 |
| D4 | n/a | >1.4 |
| D5 | >6.0 | >1.5 |
| D6 | >5.2 | n/a |

TABLE 28
Antiviral activity against Influenza on
thermoplastic polyurethane squares.

| Virus | Log reduction |
|---|---|
| Influenza | >4.0 |
| Adenovirus | >2.3 |

### Example 8. Coating Coverage, Stability, and Durability on Hands of a Volunteer

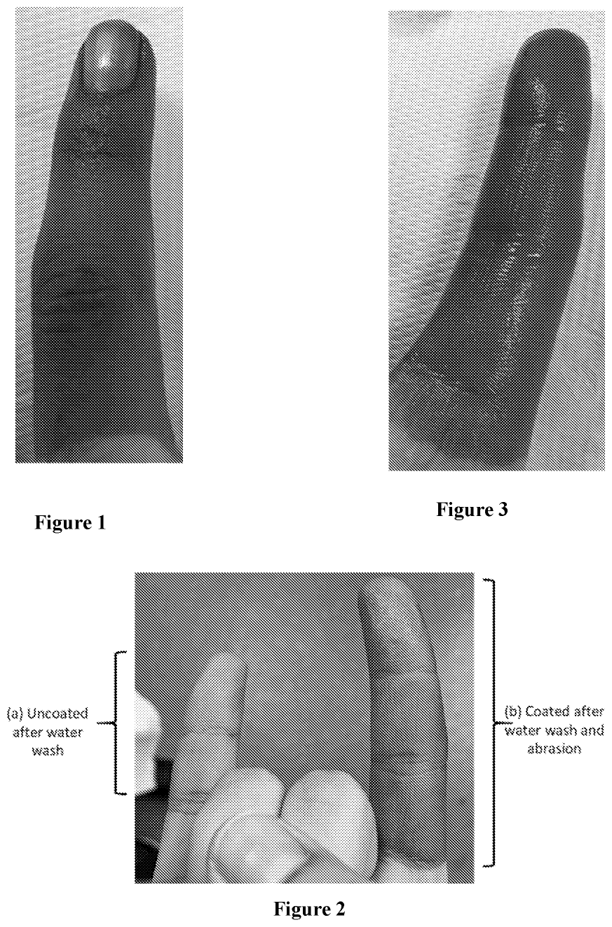

The assessment of the hand sanitiser coverage was carried out on the hands of a volunteer. This method involved spraying the hands with a sanitizer composition according to the present disclosure and left to air dry. After sufficient air drying, a dye test was carried out to show the coverage of the formulation. An example of a dyed treated index finger of the volunteer is shown in FIG. 1. A deep red colouration showing the presence of the formulation is observed.

The stability of the hand sanitizer formulation was tested by washing the treated index finger with water and applying abrasion by rubbing the index finger. The stability of the coating is shown by the red colouration remaining on the surface of the index finger after water wash and abrasion. A comparison of the treated and untreated finger after water wash and abrasion is shown in FIG. 2.

The stability of the coating was also tested by using artificial sweat to demonstrate real world application and use. The index finger was submerged in artificial sweat solution for 2 minutes before carrying out the dye test followed by water wash and abrasion. An example of a dyed untreated and treated finger after water wash and abrasion is shown in FIG. 3.

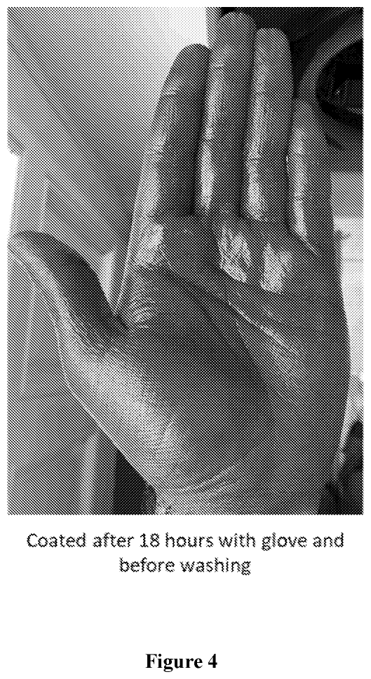

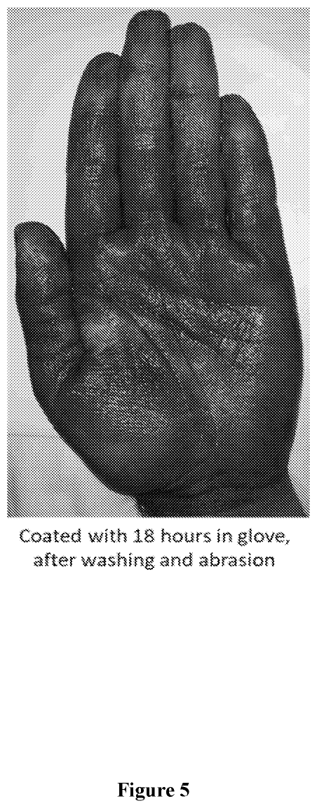

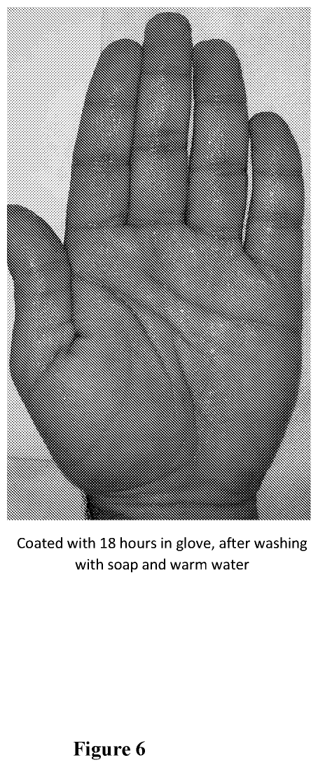

The durability of the hand sanitizer formulation was tested by applying the formulation onto one hand. The hand was then placed in a glove and left for 18 hours. After the 18 hours, the hand was subjected to the dye test as shown in FIG. 4. The stability of the coating was also tested by washing the hand with water and carrying out abrasion which is shown in FIG. 5. The stability of the formulation was strong and could be only removed after washing with soap and warm water as shown in FIG. 6.

### Example 9: Coating Coverage, Stability, and Durability on TPU Squares and Surgical Masks

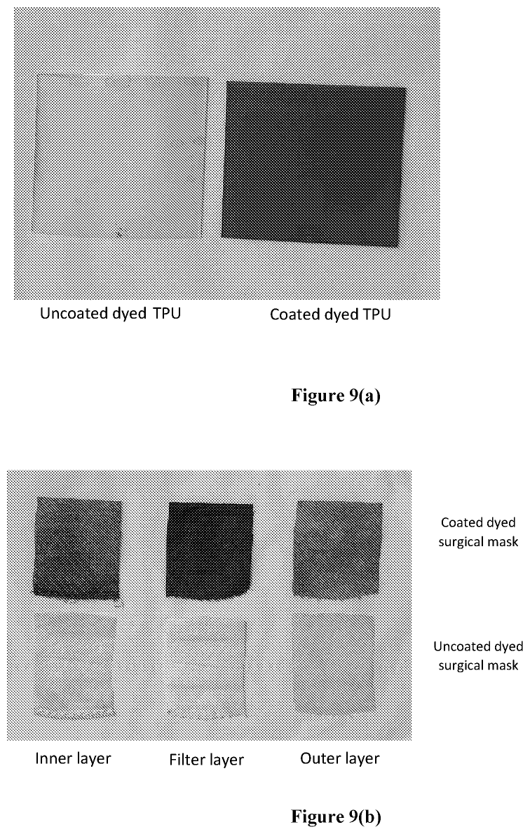

The assessment of the coating coverage on any substrate material is shown by carrying out a dye test. The test involves using a dye that binds to positively charged ions, thereby leaving a deep red colouration on the surface of the material. A substrate (TPU strip, surgical mask) coated with composition D1 in the manner herein disclosed is submerged in Ponceau Red dye and then washed with Millipore water. In the case of the TPU strip the sample undergoes abrasion. Alternatively, the coated TPU sample first undergoes wet abrasion, followed by dye. The coating coverage on the dyed surgical mask and on the dyed TPU strip following abrasion is shown by comparison with the dye coverage of an uncoated substrate—see FIG. 9. As shown in FIG. 9, the uncoated substrates show almost no uptake of dye; in comparison with the coated substrates which show a deep red and consistent colouration. This shows that the coating is still present after abrasion.

### Example 10: PAS 2424 Abrasion Testing

PAS 2424 specifies a test method for the residual bactericidal and/or yeasticidal activity of liquid chemical disinfectant products that are applied to hard, non-porous surfaces likely to undergo abrasive action. The PAS 2424 abrasion test involves performing 3 dry abrasions and 3 wet abrasions sequentially using the following procedure.

Single abrasion cycle (1 dry abrasion+1 wet abrasion):
a. Dry abrasion:
   i. A wipe is wrapped smoothly around the lid of a weighted 50 mL Falcon tube (210.0 g±2 g) and secured.
   ii. The samples are held in place and the abrasion is performed by passing the wrapped weighted Falcon tube across the surface of the sample in a forward motion followed by a backward motion. This is counted as 1 dry abrasion.
b. Wet abrasion:
   i. From a distance (˜75 cm), the wrapped weighted Falcon tube is sprayed twice with sterile water.
   ii. The abrasion is carried out using the same process as used for the dry wipe.

The above process is repeated for the 2^{nd} and 3^{rd} cycle.

### ISO 22196 and PAS 2424 Abrasion on Polyurethane Squares

This test is for determining that the coating is stable and efficacious on TPU following abrasion cycle according to the PAS 2424 procedure. The abrasion cycle is mimicking “rubbing” of the surface.

The antibacterial activity of Composition D1 on polyurethane squares after abrasion was measured using the ISO 22196 test against E. Coli. The samples were coated and then underwent three dry and wet abrasion cycles in series as per PAS 2424 procedure. Then the antibacterial activity was determined according to the ISO 22196 procedure. The results are shown in Table 29.

The results show that there was residual antibacterial activity on the surface following abrasion, demonstrating the stability of the coating.

TABLE 29
Antibacterial activity against E. Coli on
polyurethane squares after PAS 2424 abrasion.

| Composition | Log reduction |
|---|---|
| D1 | >6.21 |

### Example 11: Touch Clean Effect

Modifed EN 13697 (Bacteria)+Modified EN 16777 (Virus) with Treated Gloves

This test was conducted to demonstrate that a glove coated with an anti-microbial coating according to this disclosure is capable of disinfecting a contaminated surface. The antimicrobial activity results show (Table 30 and 31) that the treated gloves are disinfecting the inoculated stainless-steel disc, thereby disinfecting the surface.

TABLE 30
EN 13697 (gloves) bacteria E. coli.

| | Stainless steel disc |
|---|---|
| Gloves Log reduction | Log reduction |
| 1.99 | 1.00 |

TABLE 31
EN 16777 (gloves) bacteriophage Phi6.

| | Stainless steel disc |
|---|---|
| Gloves Log reduction | Log reduction |
| 4.38 | 4.00 |

Modified EN 13697 with EN 1500 (Touch Clean Hand Test)

This test was conducted to demonstrate that a treated hand is capable of disinfecting a surface contaminated with E. coli. The results below show that the treated hands are disinfecting the inoculated stainless-steel disc, thereby disinfecting the contaminated surface. This is an important and unique benefit of the anti-microbial formulation when applied to the surface of hands; showing that the treated hands are capable of disinfecting surfaces by contact.

TABLE 32
EN 13697 (hands) bacteria E. coli.

| Hands | Stainless steel disc |
|---|---|
| Log reduction | Log reduction |
| 2.5 | 1.02 |

### Example 12: Longevity Test

Longevity Sneeze Test Against E. coli and Bacteriophage Phi6 on a Porous and Non-Porous Material

This test is based on a modified ISO 22196 (bacteria nonporous)/IS021702 (virus nonporous) and ISO 20743 (bacteria porous)/ISO 18184 (virus nonporous), where sections of uncoated and coated TPU films and surgical mask were cut into 15×15 cm sections. The TPU films and surgical mask were coated with composition D5 according to this disclosure. This test was conducted to demonstrate the continuous antimicrobial activity of the coating when sprayed with pathogens over a period of 2, 3, 4 and 8 days. Each sample (coated and uncoated) was sprayed consecutively with an inoculum of 10⁹ of either bacteria or bacteriophage over 2, 3, 4, and 8 days. After inoculation, each sample was cut and placed in a sampling fluid of the relevant media for the bacteria and the bacteriophage and vortexed. Serial dilutions of the sampling fluid for the uncoated and coated samples were performed and quantified. The antimicrobial activity was determined by comparing the number of pathogens on the uncoated samples against the coated samples.

Surgical mask sections and TPU films were inoculated with the relevant pathogen and the antimicrobial activity quantified over 2,3,4 and 8 days. The results are presented in Table 33 for E. Coli and Table 34 for Bacteriophage Phi6, respectively.

The results show that the anti-microbial efficacy remains over prolonged exposure to both E. Coli and bacteriophage Phi6. Therefore, the coating has long-lasting anti-microbial residual effect demonstrating that the efficacy remains even when subjected to consecutive exposure to pathogens over several days.

TABLE 33
Antibacterial activity against E. Coli on porous
and non-porous materials over prolonged exposure.

Log reduction

| Days | Mask | TPU film |
|---|---|---|
| 2 | >4.2 | >5.5 |
| 3 | >4.9 | >4.5 |
| 4 | >3.7 | >4.4 |

TABLE 34
Antiviral activity against Bacteriophage phi6 on porous
and non-porous material over prolonged exposure.

Log reduction

| Days | Mask | TPU film |
|---|---|---|
| 2 | >6.3 | >5.0 |
| 3 | >5.1 | >5.0 |
| 4 | >7.6 | >7.5 |
| 8 | >5.8 | >7.2 |

### Example 13: Measurement of Co-Efficient of Friction

This test was to determine the dynamic coefficient of friction (CoF) of a surface when coated with and without alkyl urea polyalkylene imine polymer as disclosed herein. This was done by using a coefficient of friction machine, where thermoplastic polyurethane (TPU) strips were coated or left uncoated, and then underwent 20 measurement cycles at a lateral force of 1 Newton. The TPU strips were fixed to the coefficient of friction machine and two clamps were used to apply an even force of 1 Newton to the surface of both sides of the TPU strips. The strips were immersed in water and then pulled up through the clamps by the machine to give the dynamic coefficient value in a given area on the strip.

The results show the lubricity, stability, and durability of the coating on a surface with and without the inclusion of the alkyl urea polyalkylene imine, and when layer-coated or when coated as a blend, in accordance with this disclosure. The measured values show how much friction is generated on the surface of the TPU when an equal amount of force is applied to both sides. The lower the measured value, the lower the amount of friction generated in that given area. This thereby shows the lubricity of the coating. If the dynamic coefficient value remains constant throughout the 20 cycles this shows the durability and stability of the coating as the coating still remains on the surface of the TPU when a continuous amount of force is applied.

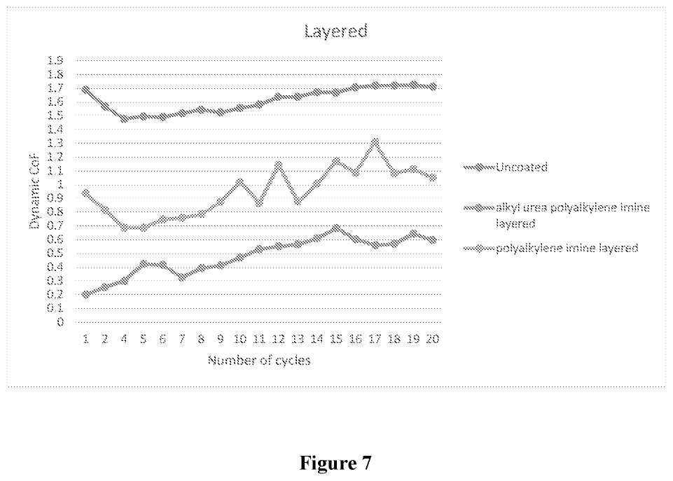

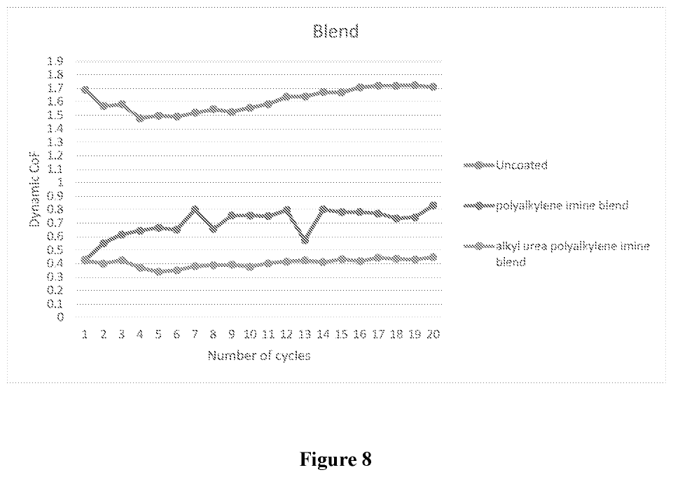

The test was carried out on TPUs where the coating was applied using either the layered or blended coating methods as disclosed herein, and compared with uncoated strips. In the layered coating method, layers of coating solutions comprising either alkyl urea polyalkylene imine or unsubstituted polyalkylene imine were applied, where the alkyl urea polyalkylene imine or the unsubstituted polyalkylene imine was present in all the applied layers. In the blended coating method, either an alkyl urea polyalkylene imine composition (composition D4) or an unsubstituted polyalkylene imine composition (a composition corresponding to D4, where the alkyl urea polyalkylene imine is replaced by unsubstituted polyalkylene imine) was applied as a blend. The results of both coating methods are presented in FIGS. 7 and 8.

FIG. 7 is a graph showing the results from TPU strips coated with the layered formulations with and without the alkyl urea polyalkylene imine. In this graph, the upper line shows the dynamic CoF values for an uncoated strip. The uncoated strip had the highest dynamic CoF value (1.4-1.7) throughout the 20 cycle and was used as a control. The lowest line on the graph shows the CoF values for the strip layer-coated with alkyl urea polyalkylene imine; whilst the middle line on the graph shows the CoF values for the strip layer-coated with unsubstituted polyalkylene imine. The formulation that had the lowest dynamic CoF was the layered formulation with the alkyl urea polyalkylene imine, and the values remained fairly constant throughout the 20 cycles. The dynamic CoF of the surface coated with alkyl urea polyalkylene imine ranged between 0.2-0.6, whereas the dynamic CoF of the surface coated with unsubstituted polyalkylene imine ranged from 0.7-1.3. Dynamic CoF values of 1 and above are not considered to be very lubricious even though this value is still lower than what was measured for the uncoated strip.

The data shows that the coatings with alkyl urea polyalkylene imine are more lubricious than the coatings with unsubstituted polyalkylene imine. The inclusion of the alkyl urea polyalkylene imine clearly has a significant impact on the lubricity of the coating, decreasing the CoF by 0.5-0.7. The graph also shows that the measured dynamic CoF values are more consistent with coatings containing alkyl urea polyalkylene imine as compared with coatings containing unsubstituted polyalkylene imine showing that the alkyl urea polyalkylene imine coatings are able to maintain lubricity over a constant force of abrasion. In comparison the unsubstituted polyalkylene imine coatings showed a much larger range of values, showing that the coating was not able to withstand the force of abrasion on the surface as well as the alkyl urea PAI coated surface. This is especially seen after 10 cycles where the value keeps on increasing and the variation gets larger with the coated unsubstituted polyalkylene imine. These results show that the alkyl urea polyalkylene imine coating is more stable and durable than the unsubstituted polyalkylene imine coating as it is able to withstand the constant force of pressure applied to the surface and the coating still remained entirely on the surface.

FIG. 8 is a graph showing the results from TPU strips coated with the blend formulations with and without the alkyl urea polyalkylene imine. In this graph, the upper line shows the dynamic CoF values for an uncoated strip. The uncoated strip was used as a control. The lowest line on the graph shows the CoF values for the strip coated with a blend formulation containing alkyl urea polyalkylene imine; whilst the middle line on the graph shows the CoF values for the strip coated with a blend formulation containing unsubstituted polyalkylene imine. Similar to the layered formulation, the strip coated with the blended alkyl urea polyalkylene imine formulation had the lowest CoF value, and this was significantly lower than CoF values for the strip coated with the blended unsubstituted polyalkylene imine formulation. The dynamic of CoF of the strip coated with alkyl urea polyalkylene imine had a constant value of around 0.4 across the 20 cycles, whereas the dynamic of CoF for the strip coated with unsubstituted polyalkylene imine ranged from 0.4-0.8. The results show that the alkyl urea polyalkylene imine blend provides a more lubricious coating than the unsubstituted polyalkylene imine blend. The blended alkyl urea polyalkylene imine coating has a constant straight line of values remaining at 0,4 across the full cycle, whilst the measurements for the blended unsubstituted polyalkylene imine coating had a larger variation of result. These measurements show that the blended alkyl urea polyalkylene imine coating is more stable and durable than the blended unsubstituted polyalkylene imine coating as it is able to withstand the applied constant force of pressure and the coating still remained entirely on the surface.

### Example 14: Competitive Testing; EN 14476 Virus Bacteriophage MS2 Suspension Test Comparing a Competitor Quaternary Ammonium Compound Hand Sanitizer (Competitor 1)

Aim: The aim of this test was to compare the antiviral activity of a hand sanitizer formulation according to this disclosure with a competitor quaternary ammonium compound product.

A viral suspension of bovine solution (3 g/l) and bacteriophage MS2 (10⁸ pfu/ml) is made in Medium 271. This inoculation suspension is added to both products. The test mixture is vortexed immediately on addition of both products and left for 2 minutes contact time. After the contact time, serial dilutions of both testing mixture are made and plated out on the Medium 271 plates. The Agar plates are incubated at 37° C. for approximately 12-24 hours.

TABLE 35
Result from EN 14476 suspension test.

| Formulations | Log reduction | |
|---|---|---|
| Competitor 1 - (quaternary ammonium compound) | >0.02 | log |
| Hand sanitizer | >2 | log |

In this test the hand sanitizer of this disclosure showed strong antiviral activity against non-enveloped virus bacteriophage MS2 at a short time period of 2 minutes, as shown in Table 35. By comparing the two products, the hand sanitizer gained a significant log reduction, whilst Competitor 1 had little to no activity against the non-enveloped virus which is also reflected in the percentage reduction. This shows that the hand sanitizer of the current disclosure is capable of killing non-enveloped viruses whilst Competitor 1 cannot, as its log and percentage reduction is mostly likely due to variation in the test itself and not the product, which means the hand sanitizer is effective against non-enveloped viruses and Competitor 1 is not.

### Example 15: Competitive Testing; 24 Hours Pig Skin Study Unwashed and Washed Against E. coli and MS2 Comparing a Competitor Quaternary Ammonium Compound Hand Sanitizer (Competitor 1)

Aim: The aim of this test was to show the residual antimicrobial activity of both product formulations after being applied 24 hours onto pig skin and undergoing a water wash procedure.

Samples from pig skin were cut to a desired area (2.25 cm²). The samples were then submerged in Millipore water and IPA to remove any excess salt and to sterilise the samples for the antimicrobial testing. Both Competitor 1 and the hand sanitizer formulation were then applied to the pig skin samples by submerging the pig skin for 1 minute in 100 ml volume of each formulation. After the 1-minute dip coating, uncoated and coated samples were then placed into petri dishes and sealed with tape, which were then placed in boxes to be left for 24 hours. After 24 hours, half of the coated samples underwent a washing procedure to show the residual activity of each product when applied to skin. Once all the samples were ready each was sprayed with bacterial (E. coli)/virus (bacteriophage MS2) suspension. The samples were left for the desired contact time and quantified by transferring the pig skin samples into 10 mls of recovery media and plating serial dilution of this media.

TABLE 36
Results from 24 hours pig skin test against E. coli.

| Formulations | Log reduction |
|---|---|
| Competitor 1 - (quat ammonium compound) unwashed | no reduction |
| Competitor 1 - (quat ammonium compound) washed | no reduction |
| Hand sanitizer unwashed | >1.6 log |
| Hand sanitizer washed | >1.3 log |

TABLE 37
Results from 24 hours pig skin test against bacteriophage MS2.

| Formulations | Log reduction |
|---|---|
| Competitor 1 - (quat ammonium compound) unwashed | >0.26 log |
| Competitor 1 - (quat ammonium compound) washed | >0.23 log |
| Hand sanitizer unwashed | >1.21 log |
| Hand sanitizer washed | >1.01 log |

In this test the hand sanitizer showed strong antimicrobial activity against both a non-enveloped virus (bacteriophage MS2) and a gram-negative bacterium (E. coli) after staying on skin for 24 hours, as shown in Tables 36 and 37. It also shows that the hand sanitizer has residual activity even after the washing procedure as its log reduction did not change significantly against both microorganisms. This proves that the protection from the hand sanitizer was maintained for 24 hours and was able to withstand the washing regime. When comparing against Competitor 1 little to no activity against both microorganisms was observed, proving that this product does not have antimicrobial activity after 24 hours, nor able to withstand the washing regime. This is reflected in the log reduction which is most likely due to variation in the test itself and not the product.

### Example 16: Competitive Testing; EN 1500 Hand Test with Washing Against E. coli

Aim: The aim of this test was to show the residual antimicrobial activity of both product formulations when applied to hands of a volunteer whilst also carrying out a water wash procedure.

For this test untreated hands are compared against hands that were also treated with Competitor 1 and the hand sanitizer product, that underwent a washing procedure.

### 1) Inoculation

First the volunteer washed their hands with soap to get rid of any residual flora present. Once washed and dried, the volunteer was sprayed with the bacteria (E. coli)/virus (bacteriophage MS2) suspension and left to dry for 1 minute.

### 2) Sampling

After 1 minute drying time the fingertips of were submerged in 10 ml of the relevant media and used to quantify the amount of inoculum present, by plating out serial dilutions of this sampling fluid.

3) Applying Products before wash

Before applying the individual products, the hands of the volunteer underwent the inoculation process. Once the hands were inoculated, a certain volume of each formulation was then applied and rubbed across the hands of the volunteer. The sampling process was then carried out.

4) Testing product after water wash

During this produce the hands of the volunteer were first applied with each formulation using the same volume as before. Once applied they were left to dry for 5 minutes. The volunteer then underwent a washing procedure and carried out the inoculation and sampling processes.

TABLE 38
Results from EN 1500 hand test against E. coli.

| | Left hand | | Right hand | |
|---|---|---|---|---|
| Formulation | Log reduction | | Log reduction | |
| Competitor 1 - (quaternary ammonium | >1.26 | log | >1.66 | log |
| compound) unwashed | | | | |
| Competitor 1 - (quaternary ammonium | no reduction | | >0.17 | log |
| compound) washed | | | | |
| Hand sanitizer unwashed | >5.36 | log | >5.6 | log |
| Hand sanitizer washed | >4.2 | log | >4.0 | log |

TABLE 39
Results from EN 1500 hand test against bacteriophage MS2.

| | Left hand | Right hand |
|---|---|---|
| Formulation | Log reduction | Log reduction |
| Competitor 1 - (quaternary ammonium | >0.32 log | >0.40 log |
| compound) unwashed | | |
| Competitor 1 - (quaternary ammonium | >0.45 log | >0.38 log |
| compound) washed | | |
| Hand sanitizer unwashed | >2.11 log | >1.77 log |
| Hand sanitizer washed | >1.27 log | >1.00 log |

In this test the hand sanitizer showed strong antimicrobial activity against bacteriophage MS2 and E. coli as shown in Tables 38 and 39. It also shows that the antimicrobial activity was still significant to provide protection against both microorganisms even after performing the washing procedure. When comparing against Competitor 1 some activity against E. coli was seen. This antimicrobial activity against E. coli was not seen after performing the washing procedure therefore suggesting that this product was mostly likely rubbed off when carrying out this process. Again, Competitor 1 showed little to no activity against bacteriophage MS2, even when it was applied immediately after inoculation.

### Conclusion from Examples 14-16

All the tests above were carried out on both Competitor 1 (quaternary ammonium compound) and the hand sanitizer formulation according to this disclosure. Each product claims to have long lasting protection on skin whilst having broad spectrum activity. The suspension tests only show the strength of the antimicrobial activity of each formulation, but do not reflect the protection each product can provide when applied to skin. In the EN 14476 test, the hand sanitizer showed a strong antimicrobial activity against non-enveloped virus. Competitor 1 did not have any antiviral activity against a non-enveloped virus in the EN 14476 test.

During the pig skin test mentioned above, the antimicrobial activity was tested after 24 hours whilst the water wash procedure was used to test for residual antimicrobial activity. These tests simulate the real-world application for these products as they demonstrate the protection of the hand sanitizer over a prolong period of time whilst showing that this protection can last the harsh environments that they will be used in.

The 24 hours pig skin test demonstrates the ability of the disclosed formulation to deliver 24-hour protection whilst also showing its residual activity. During this test Competitor 1 was not able to show any significant antimicrobial activity against Bacteriophage MS2. Against bacteria Competitor 1 was able to show some antimicrobial activity; however this was lost after the washing regime, therefore this product will not be able to provide protection for 24 hours. The hand sanitizer of this disclosure showed a significant log reduction after 24 hours even under washing, which was at least 5 times greater log reduction compared to Competitor 1 showing that this product has long lasting protection and can withstand the washing procedures.

The EN 1500 hand test demonstrates each product when applied to volunteers and their residual antimicrobial activity. When tested against E. coli both products showed antibacterial activity on the unwashed procedure, however the hand sanitizer activity was on average 4 times greater in log reduction on both hands than compared to Competitor 1. After the washing procedure the hand sanitizer of this disclosure was able to maintain a strong antibacterial activity whilst Competitor 1 showed no reduction therefore showing that the product had been washed off and that the product does not work when tested in a real-world application. When tested against bacteriophage MS2 the hand sanitizer of this disclosure was able to show a maintained antiviral activity when comparing before and after washing, whilst Competitor 1 showed very little to no activity.

### Example 17: Coating Surface Test Combining PAS 2424+ISO 22196/ISO 21702 on Thermoplastics Polyurethane (TPU)

Aim: The aim of this test was to show the residual antimicrobial activity of a surface coating according to this disclosure and Competitor 1 surface spray (quaternary ammonium compound) when applied to a non-porous surface.

For this test both products were applied to TPU surface using a dip coating method. Once applied, each product underwent 3 cycles of dry and wet abrasion using polyester wipes wrapped around a weight. Both un-abrased and abrased samples were inoculated with the bacterial (E. coli)/virus (bacteriophage MS2) suspension and covered with a thin film to ensure the same surface area was inoculated across all the samples. These samples were then left for the desired contact time and then transferred into 10 mls of relevant media to quantify the amount of inoculum left on each sample by plating out serial dilutions of this sampling fluid.

TABLE 40
Results from PAS 2424 and ISO 22196 test (E. coli).

| Formulations | Log reduction |
|---|---|
| Competitor 1 - (quaternary ammonium compound) | >0.13 log |
| Competitor 1 - (quaternary ammonium compound) PAS | >0.17 log |
| 2424 abrased | |
| Surface coating | >2.19 log |
| Surface coating PAS 2424 abrased | >2.05 log |

TABLE 41
Results from PAS 2424 and ISO 21702 test (bacteriophage MS2).

| Formulations | Log reduction |
|---|---|
| Competitor 1 - (quaternary ammonium compound) | >0.21 log |
| Competitor 1 - (quaternary ammonium compound) PAS | >0.13 log |
| 2424 abrased | |
| Surface coating | >1.17 log |
| Surface coating PAS 2424 abrased | >1.08 log |

In this test the surface coating of this disclosure showed strong antimicrobial activity against bacteriophage MS2, and E. coli as shown in Tables 40 and 41. This antimicrobial activity of the surface coating of this disclosure still remained on the TPU surface after the wet and dry abrasion cycles demonstrating a strong durability and stability of the coating. When compared against Competitor 1 this had little to no activity against both microorganisms on both the un-abrased and abrased samples. This suggest that Competitor 1 is unable to kill microorganisms on non-porous surfaces, therefore will not provide long lasting protection.

### Example 18: Coating Surface Test ISO 20743/ISO 18184 on Surgical Mask with Washing Procedure

Aim: The aim of this test was to show the residual antimicrobial activity of a surface coating according to this disclosure and Competitor 1 surface spray when applied to porous surface after water wash procedure.

For this test both products were applied to porous surgical mask samples using a dip coating method. Once applied to the porous surface each product underwent a water wash procedure and the dried before testing. Both un-washed and washed samples were inoculated with the bacterial (E. coli)/virus (bacteriophage MS2) suspension. These samples were then left for the desired contact time and then transferred in 20 ml of relevant media to quantify the amount of inoculum left on each sample by plating out serial dilutions of this sampling fluid.

TABLE 42
Results from ISO 20743 (E. coli) test with and without washing.

| Formulations | Log reduction |
|---|---|
| Competitor 1 - (quaternary ammonium compound) - | >4.3 log |
| unwashed | |
| Competitor 1 - (quaternary ammonium compound) - | >2.02 log |
| washed | |
| Surface coating - unwashed | >4.3 log |
| Surface coating PAS 2424 - washed | >4.3 log |

TABLE 43
Results from ISO 18184 (bacteriophage
MS2) test with and without washing.

| Formulations | Log reduction | |
|---|---|---|
| Competitor 1 - (quaternary ammonium compound) - | >0.41 | log |
| unwashed | | |
| Competitor 1 - (quaternary ammonium compound) - | >0.42 | log |
| washed | | |
| Surface coating - unwashed | >3.3 | log |
| Surface coating - washed | >3.1 | log |

In this test both Competitor 1 and the surface coating of this disclosure (unwashed samples) showed strong antimicrobial activity against E. coli as shown in Table 42. This reduction was the same for the surface coating of the disclosure after the washing procedure, thereby demonstrating that the coating remained on the mask. However, the antimicrobial activity of Competitor 1 decreased by 2 logs thereby showing that the product was removed during the washing produce and is not as durable or stable as the surface coating when applied to porous surfaces. The results from Table 43 show that the surface coating of this disclosure has a strong antiviral activity against the non-enveloped virus bacteriophage MS2, whilst Competitor 1 showed no activity. The log reduction from Competitor 1 is negligible and mostly due to variation in the test and not from the product. The log reduction was unchanged the surface coating of the disclosure after the washing procedure, thereby demonstrating that the coating remained present and active on the mask.

### Example 19: Coating Surface Test ISO 18184 on Surgical Mask

Aim: The aim of this test was to show the antimicrobial activity of the surface coating of this disclosure when compared to Competitor 2 (silver technology).

For this test the surface coating was applied to a surgical mask and compared against Competitor 2 mask which had been treated with silver technology. Both un-treated and washed samples were inoculated with the virus (bacteriophage MS2) suspension. These samples were then left for the desired contact time and then transferred in 20 mls of relevant media to quantify the amount of inoculum left on each sample by plating out serial dilutions of this sampling fluid.

TABLE 44
Results from ISO 18184 (bacteriophage MS2) test.

| Formulations | log reduction |
|---|---|
| Competitor 2 (silver technology) | >0.01 log |
| Surface coating | >2.90 log |

The results from Table 44 show that the surface coating of this disclosure has a strong antiviral activity against the non-enveloped virus bacteriophage MS2, whilst Competitor 2 showed no activity. The log reduction from Competitor 2 is negligible and mostly due to variation in the test and not from the product.

### Example 20: Touch Clean Testing with Nitrile Gloves

Aim: The aim of this test was to show the unique benefit of the surface coating touch clean technology, by demonstrating the anti-microbial contact effect of a disinfectant composition according to this disclosure, in comparison with Competitor 1 (quaternary ammonium compound) surface spray.

For this test both products were applied to Nitrile gloves using the spray coating method, ensuring the whole glove was covered. In this test the bacterial (E. coli)/viral suspension (bacteriophage MS2) was made with bovine serum which was used to inoculate a stainless-steel disc and dried by placing the discs in an incubator at 37° C. Once dried, a volunteer wore the uncoated/coated gloves and using each hand placed their fingertips on top of the dried inoculum for the desired contact time. To quantify the amount of inoculum left on each surface, the stainless-steel discs were placed in 10 ml of the relevant media. The volunteer rubbed their fingertips in 10 mls of the same media at the same time. Serial dilutions of both sampling fluids were prepared and plated out onto the relevant agar plates.

TABLE 45
Results from Touch clean Nitrile glove (E. coli) test.

| | Log reduction | |
|---|---|---|
| Formulation | Stainless steel disc | gloves |
| Competitor 1 (quat ammonium | No reduction | No reduction |
| compound) | | |
| Surface Coating | >1.57 log | >1.27 log |

TABLE 46
Results from Touch clean Nitrile glove (bacteriophage MS2) test.

| | Log reduction | |
|---|---|---|
| | Stainless steel | |
| Formulation | disc | Gloves |
| Competitor 1 (quat ammonium compound) | >0.06 log | No reduction |
| Surface Coating | >1.03 log | >0.97 log |

This test shows the unique benefit of the surface coating touch clean technology. The surface coating of this disclosure showed antiviral activity against bacteriophage MS2 and E. coli on both the gloves and stainless-steel disc. Competitor 1 did not show any reduction when tested against E. coli and negligible reduction against bacteriophage MS2 on either surface. This shows that the surface coating when applied to gloves is able to disinfect the contaminated stainless-steel surface whilst also reducing the transmission of microorganisms to the gloves itself. Competitor 1 did not disinfect the stainless-steel surface or prevent the transmission of both bacteriophage MS2 and E. coli to the gloves.

### Conclusion from Examples 17-20

All the tests above were carried out on the surface coating against either Competitor 1 (quaternary ammonium product) or 2 (chlorhexidine silver product). Each product claims to have long lasting protection on surfaces whilst having broad spectrum activity. PAS 2424 abrasions were used to simulate high contact areas as these surfaces will undergo a high number of rubbing throughout their functional lifetime. The water wash procedure used on the mask was to show that the products could withstand a washing procedure and therefore allow these masks to be reusable. These tests simulate the real-world application for these products as they demonstrate the protection of a non-porous and a porous surface over a prolonged period of time by surviving the harsh environments in which they will be used.

When carrying out the PAS 2424 tests on a non-porous surface, Competitor 1 was not able to show any antimicrobial activity either before or after abrasion. This shows that the product was not able to provide long lasting protection. Overall, against both microorganisms the surface coating had a strong antimicrobial activity where the log reduction was on average 14 times greater than Competitor 1 for both un-abrased and abrased surfaces against E. coli and 6 times greater than Competitor 1 against bacteriophage MS2.

The first porous antimicrobial test was carried out on both the surface coating and Competitor 1 when applied to surgical mask and tested against E. coli and bacteriophage MS2. Both un-washed and washed samples were tested in order to demonstrate long lasting protection on reusable porous surface (e.g. surgical mask, gowns, and tabletops). These products will need to withstand washing regimes as these surfaces can be spoiled when in use, therefore will be washed to remove any stains (such as blood) on the surface. Against E. coli, both the surface coating and Competitor 1 had strong antibacterial activity on the unwashed samples, however for Competitor 1 this activity did drop to 2 logs on the washed samples and did not show any significant activity against MS2. The surface coating was able to maintain its protection against both microorganisms after the washing procedure and had a log reduction 2 times greater than Competitor 2 when tested against E. coli. The results shows that the surface coating has a greater durability and stability than Competitor 1, and therefore will have a longer lasting protection on porous surface, whist Competitor 1 protection will be removed during washing. The second porous antimicrobial test was carried out with both the surface coating and Competitor 2. This silver technology has been known to kill a number of viruses which is why it was compared to the surface coating. Competitor 2 did not show any antiviral activity against bacteriophage MS2, showing that it cannot kill non-enveloped viruses, whilst the surface coating showed a strong antiviral activity.

Touch Clean technology is a unique benefit of the disclosed antimicrobial formulations. This technology was demonstrated on the nitrile gloves and compared against Competitor 1 to show that this technology is unique to the surface coating and cannot be seen from other products. This unique ability allows the surface coating to disinfect surfaces that it comes into contact with i.e. contaminated surfaces. During these tests not only was the surface coating able to disinfect the contaminated surface but also reduce the transmission of microorganism to the nitrile glove. This technology is especially relevant to highly contaminated area such as the healthcare facility and allows staff to reduce transmission to patient and vice versa whilst also maintaining a healthy environment.

### Example 21: Zone of Inhibition Assay Illustrates Non-Leaching Quality of Coating

For this test uncoated TPU's (negative control), coated TPU's (tested samples), with dimension of 5 mm×5 mm were used as well as a positive control catheter (chlorhexidine/silver). In this test TPU strips were coated either with composition C2 or with composition D1, or were left uncoated (control). E. coli was grown overnight in TSB broth at 370° C. After incubating overnight, the turbidity of the bacterial culture was measured and adjusted to 1×10⁷ CFU/ml in TSB. Using the bacterial suspension 500 μl was transferred to a TSA plate and using a blue loop the suspension was spread evenly over the surface. The TSA plates were dried for 30 minutes to allow the bacterial lawn to “settle” and any excess liquid was drawn into agar. Once the TSA plate had been dried using sterile tweezers the 5 mm×5 mm samples were forced and penetrated into the TSA plates ensuring that the sample reached the bottom of the petri dish and was in full contact with the bacterial lawn. The TSA plate was incubated at 37° C. for 18-24 hours. The next day the plates were removed from the incubator and any zone of inhibition present was measured.

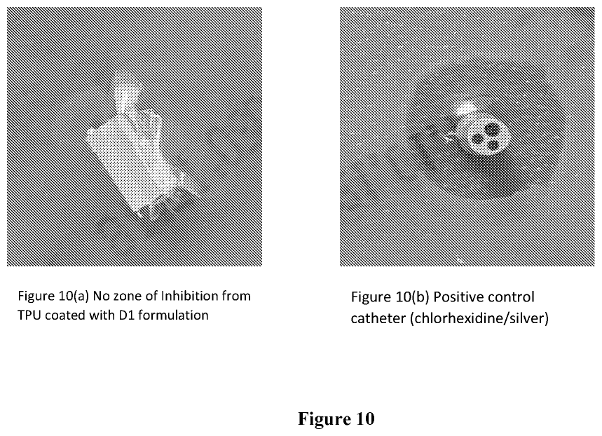

The results from the zone of inhibition test are shown below in Table 47. FIG. 10(a) illustrates a TPU strip coated with composition D1 showing no zone of inhibition, hence no leaching; and the positive control, catheter coated with chlorhexidine/silver—showing a zone of inhibition which provides evidence of leaching.

TABLE 47
Area of inhibition from the TPU samples.

| Sample | Area of inhibition (cm²) |
|---|---|
| Uncoated TPU | No zone of inhibition |
| C2 | No zone of inhibition |
| D1 | No zone of inhibition |
| Positive control catheter (chlorhexidine/silver) | 1.76 |

1-63. (canceled)

64. A liquid coating composition comprising an alkyl urea polyalkylene imine polymer and an anionic polymer; wherein the liquid coating composition is suitable for forming an anti-microbial coating.

65. A liquid coating composition as claimed in claim 64, which is formulated as a solution, suspension, dispersion, or emulsion, in a liquid medium which is aqueous, alcoholic, aqueous/alcoholic, or organic solvent.

66. A liquid coating composition as claimed in claim 64, further comprising one or more additional cationic polymers, such as a polyalkylene imine polymer which is not an alkyl urea polyalkylene imine polymer, such as an unsubstituted polyalkylene imine polymer or an alkylated polyalkylene imine polymer.

67. A liquid coating composition as claimed in claim 64, further comprising a guanidine compound, wherein the guanidine compound comprises a cross-linkable polymer, and wherein the liquid coating composition further comprises a cross-linking agent for cross-linking the polymer.

68. A liquid coating composition as claimed in claim 67, wherein the cross-linkable polymer bears one or more cross-linkable carboxylic acid groups.

69. A liquid coating composition as claimed in claim 64, comprising at least about 0.005% w/v of said alkyl urea polyalkylene imine polymer, and/or at least about 0.1% w/v of total cationic polymers including said alkyl urea polyalkylene imine polymer and any additional cationic polymer(s).

70. A liquid coating composition as claimed in claim 64, comprising no more than about 25% w/v of said alkyl urea polyalkylene imine polymer, or comprising no more than about 25% w/v of total cationic polymers including said alkyl urea polyalkylene imine polymer and any additional cationic polymer(s).

71. A liquid coating composition as claimed in claim 64, comprising between about 0.01-7% w/v of said alkyl urea polyalkylene imine, and optionally further comprising 0.01-10% w/v of additional cationic polymer(s).

72. A liquid coating composition as claimed in claim 64, comprising an alkyl urea polyalkylene imine and one or more additional cationic polymer(s) in a w/w ratio in the range 1:50-5:1.

73. A liquid coating composition as claimed in claim 64, comprising at least about 0.001% w/v of said anionic polymer and/or comprising no more than about 0.5% w/v of said anionic polymer; or comprising between about 0.001-0.3% w/v of said anionic polymer.

74. A liquid coating composition as claimed in claim 64, wherein the w/w ratio of the combined quantity of alkyl urea polyalkylene imine and any additional cationic polymer(s) anionic component is in the range of 500:1-15:1.

75. A liquid coating composition as claimed in claim 64, wherein the composition comprises:
(A) 0.02-4.5% w/v of said alkyl urea polyalkylene imine;
0.001-0.2% w/v of said anionic polymer; and
optionally 0.1-5% w/v of additional cationic polymer(s);
or
(B) 0.05-3.5% w/v of a guanidine compound;
0.01-7% w/v of said alkyl urea polyalkylene imine;
0.002-0.1% w/v of said anionic polymer; and
optionally 0.05-4% w/v of additional cationic polymer(s);
or
(C) 0.01-2% w/v of said alkyl urea polyalkylene imine;
0.005-0.3% w/v of said anionic polymer, such as polyacrylic acid;
0.1-1% w/v of additional cationic polymer such as polyethylene imine; and optionally
0.001-0.2% w/v of benzalkonium chloride and/or benzethonium chloride;
where the total quantity of alkyl urea polyalkylene imine, anionic component, additional cationic polymer and benzalkonium chloride and/or benzethonium chloride is 0.1-4% w/v;
or
(D) 0.05-7% w/v of said alkyl urea polyalkylene imine;
0.3-8% w/v of additional cationic polymer such as polyethylene imine;
0.5-3.5% w/v of guanidine compound; and optionally
0.001-0.2% w/v of benzalkonium chloride and/or benzethonium chloride, and/or 0.005-0.3% w/v of said anionic polymer, such as polyacrylic acid;
where the total quantity of alkyl urea polyalkylene imine, anionic polymer, additional cationic polymer, guanidine compound and benzalkonium chloride and/or benzethonium chloride is 1-19% w/v.

76. A method for preventing or reducing the growth or spread or the quantity of one or more micro-organisms on a surface of a substrate or article, and/or for inactivating one or more micro-organisms on a surface of a substrate or article, and/or for preventing the formation of and/or disrupting and/or removing a surface biofilm on a surface of a substrate or article, which method comprises applying an anti-microbial coating comprising an alkyl urea polyalkylene imine polymer and an anionic polymer to the surface, or contacting the surface with a coated substrate or article that comprises an anti-microbial coating comprising an alkyl urea polyalkylene imine polymer and an anionic polymer.

77. A method as claimed in claim 76, wherein the step of applying an anti-microbial coating to the surface involves exposing the surface to the liquid coating composition of claim 64.

78. A method as claimed in claim 77, wherein the step of exposing the surface to the liquid coating composition involves incubating the substrate or article in the liquid coating composition, and/or immersing the substrate or article in the liquid coating composition, and/or washing the substrate or article with the liquid coating composition, and/or dipping the substrate or article into the liquid coating composition either once or more than once, and/or causing the liquid coating composition to flow over the substrate or article, and/or spraying the liquid coating composition onto the substrate or article, and/or painting the liquid coating composition onto the substrate or article, and/or wiping the liquid coating composition onto the substrate or article, and/or brushing the liquid coating composition onto the substrate or article, and/or padding the liquid coating composition onto the substrate or article, and/or rolling the liquid coating compositions onto the substrate or article, and/or applying the liquid coating composition to the substrate or article using any other application technique or combination or sequence or application techniques, including physical vapor deposition and/or electrophoretic deposition; followed by an optional step (b) of drying or curing the coated substrate or article or allowing the coated substrate or article to dry or cure.

79. A method as claimed in claim 76, wherein the surface is a surface of a body part of a living human or animal; such as the skin, such as the hands or face or feet, of a human.

80. A method as claimed in claim 79, wherein the step of applying a coating to the surface of a body part of a living human or animal involves applying the liquid coating composition of claim 64 to the body part, by washing and/or rinsing the body part with the liquid coating composition and/or by spraying, rubbing, padding, rolling, depositing and/or brushing the liquid coating composition onto the body part.

81. A method as claimed in claim 79, wherein the liquid coating composition further comprises glycerol and/or benzalkonium chloride and/or benzethonium chloride, optionally in a quantity of between about 0.005-1.0% w/v, and/or is formulated for administration to the skin of a human or animal.

82. A method as claimed in claim 76, wherein the substrate or article is inert (non-living), and is formed from porous and/or non-porous materials, and/or from natural and/or artificial materials and/or biodegradable and/or non-biodegradable materials; optionally wherein the substrate or article comprises one or more of a plastic material, an elastomeric material, such as an elastane, such as Spandex® or Lycra®, or a synthetic rubber, and/or a polymeric material including a polyurethane or silicone and/or silica material and/or a natural or synthetic biopolymer or bioabsorbable material; and/or marble, stone, composite material, wood, and/or rubber; and/or a metal and/or metal alloy including stainless steel; and/or a ceramics material; and/or glass; and/or an organic material including an animal-derived material such as collagen and/or a decellularized graft; or mixtures and combinations thereof; and/or wherein the substrate or article comprises a fabric or textile, such as a woven or non-woven fabric, including natural or synthetic fibers or fabric or textile material; including a melt-blown polymer material, or nylon or rayon or polyester or polyester cellulose or polyethylene or polypropylene; and optionally wherein the substrate or article is a medical device such as a catheter or implant, including a heart valve, stent, graft and/or scaffold, or an endoscope; or a medical implement such as a dish or spatula or a surgical tool; or diagnostic equipment; or an item of personal protective equipment (PPE) such as a mask, gown including a surgical gown, face shield, medical scrubs, eye protection, or gloves; or an item for use in cleaning, purification or sterilization, such as a cloth, sponge, filter or wipe; or wherein the substrate is a surface which is regularly touched or handled by people, such as a door or a handle of a door or window, or a support or guard rail, or a surface of any object in a public or semi-public place including public transport, or in an institution including a school, college, hospital, medical center, government or local council center, courthouse or prison, or in a private or semi-private venue including a shop, entertainment center, restaurant, or private home.

83. A method as claimed in claim 76, for inactivating or preventing or reducing the growth or spread or the quantity of bacteria, including gram negative, gram positive and drug resistant bacteria; including nosocomial pathogens; including but not limited to any one or more of E. coli, S. aureus, E. hirae and P. aeruginosa strains and methicillin-resistant S. aureus (MRSA) strains; and/or viruses, including enveloped and non-enveloped viruses; including but not limited to adenovirus, norovirus, vaccinia virus and coronavirus strains; and/or fungi, such as yeast, including but not limited to C. albicans strains.
